(19) United States
(12) Patent Application Publication
(10) Pub. No.: US 2010/0311782 A1
(43) Pub. Date: Dec. 9, 2010

(54) SUBSTITUTED PIPERIDINYLPROPANOIC ACID COMPOUNDS AND METHODS OF THEIR USE

(75) Inventors: Roland E. Dolle, King of Prussia, PA (US); Bertrand Le Bourdonnec, East Fallowfield, PA (US)

Correspondence Address: FELDMAN GALE, P.A., 1700 Market Street, Suite 3130, Philadelphia, PA, 19103, US

(73) Assignee: Adolor Corporation, Exton, PA (US)

(21) Appl. No.: 12/795,095

(22) Filed: Jun. 7, 2010

Related U.S. Application Data
(60) Provisional application No. 61/184,891, filed on Jun. 8, 2009.

(51) Int. Cl. A61K 31/485; C07D 211/34; A61K 31/451; A61K 31/452; A61K 31/454; A61K 31/4748; A61K 31/4545; A61P 1/04; A61P 1/06; A61P 1/10; A61P 29/00; C12N 5/02

(52) U.S. Cl. 514/282; 546/224; 514/331; 514/326; 514/289; 514/330; 514/295; 514/318; 435/375

(57) ABSTRACT

Novel 3,4-disubstituted-4-(3-carbamoylphenyl)-piperidinylpropanoic acid compounds and their salts, including pharmaceutically acceptable salts, pharmaceutical compositions and methods of their use are disclosed. The novel compounds are useful, inter alia, as antagonists of opioid receptors.

### CROSS REFERENCE TO RELATED APPLICATIONS

This application claims the benefit of U.S. Application No. 61/184,891, filed Jun. 8, 2009, the entirety of which is hereby incorporated herein by reference.

### FIELD OF THE INVENTION

The present invention relates to substituted piperidinylpropanoic acid compounds that may affect the opioid receptor system. More particularly, this invention relates to 3,4-disubstituted-4-(3-carbamoylphenyl)piperidinylpropanoic acid compounds and their use, inter alia, as antagonists of opioid receptors.

### BACKGROUND OF THE INVENTION

It is well known that opioid drugs target three types of endogenous opioid receptors (i.e., μ, δ, and κ receptors) in biological systems. Many opiates, such as morphine, are μ opioid agonists that are often used as analgesics for the treatment of severe pain due to their activation of μ opioid receptors primarily, though not exclusively, in the central nervous system (CNS). Opioid receptors are, however, not limited to the CNS, and may be found in other tissues throughout the body, i.e., peripheral to the CNS. A number of side effects of opioid drugs may be caused by activation of these peripheral receptors. For example, administration of μ opioid agonists often results in intestinal dysfunction due to the large number of receptors in the wall of the gut (Wittert, G., Hope, P. and Pyle, D., Biochemical and Biophysical Research Communications 1996, 218, 877-881; Bagnol, D., Mansour, A., Akil, A. and Watson, S. J., Neuroscience 1997, 81, 579-591). Specifically, opioids are generally known to cause nausea and vomiting, as well as inhibition of normal propulsive gastrointestinal function in animals and man (Reisine, T., and Pasternak, G., Goodman & Gilman's The Pharmacological Basis of Therapeutics, Ninth Edition 1996, 521-555), resulting in side effects such as, for example, constipation.

Naturally-occurring endogenous opioid compounds may also affect propulsive activity in the gastrointestinal (GI) tract. Met-enkephalin, which activates μ and δ receptors in both the brain and gut, is one of several neuropeptides found in the GI tract (Koch, T. R., Carney, J. A., Go, V. L., and Szurszewski, J. H., Digestive Diseases and Sciences 1991, 36, 712-728). Additionally, receptor knockout techniques have shown that mice lacking μ opioid receptors may have faster GI transit times than wild-type mice, suggesting that endogenous opioid peptides may tonically inhibit GI transit in normal mice (Schuller, A. G. P., King, M., Sherwood, A. C., Pintar, J. E., and Pasternak, G. W., Society of Neuroscience Abstracts 1998, 24, 524). Studies have shown that opioid peptides and receptors located throughout the GI tract may be involved in normal regulation of intestinal motility and mucosal transport of fluids in both animals and man (Reisine, T., and Pasternak, G., Goodman & Gilman's The Pharmacological Basis of Therapeutics, Ninth Edition 1996, 521-555). Other studies show that the sympathetic nervous system may be associated with endogenous opioids and control of intestinal motility (Bagnol, D., Herbrecht, F., Jule, Y., Jarry, T., and Cupo, A., Regul. Pept. 1993, 47, 259-273). The presence of endogenous opioid compounds associated with the GI tract suggests that an abnormal physiological level of these compounds may lead to bowel dysfunction.

It is a common problem for patients having undergone surgical procedures, especially surgery of the abdomen, to suffer from a particular bowel dysfunction called post-surgical (or post-operative) ileus. “Ileus,” as used herein, refers to the obstruction of the bowel or gut, especially the colon. See, e.g., Dorland's Illustrated Medical Dictionary, 27th ed., p. 816, (W.B. Saunders Company, Philadelphia, Pa., 1988). Ileus should be distinguished from constipation, which refers to infrequency of or difficulty in feces evacuation. See, e.g., Dorland's Illustrated Medical Dictionary, 27th ed., p. 375, (W. B. Saunders Company, Philadelphia 1988). Ileus may be diagnosed by the disruption of normal coordinated movements of the gut, resulting in failure of intestinal contents propulsion. See, e.g., Resnick, J. Am. J. of Gastroenterology 1997, 92, 751 and Resnick, J. Am. J. of Gastroenterology, 1997, 92, 934. In some instances, particularly following surgery, including surgery of the abdomen, the bowel dysfunction may become quite severe, lasting for more than a week and affecting more than one portion of the GI tract. This condition is often referred to as post-surgical (or post-operative) paralytic ileus and most frequently occurs after laparotomy (see Livingston, E. H. and Passaro, Jr., E. D., Digestive Diseases and Sciences 1990, 35, 121). Similarly, post-partum ileus is a common problem for women in the period following childbirth, and is thought to be caused by similar fluctuations in natural opioid levels as a result of birthing stress.

Gastrointestinal dysmotility associated with post-surgical ileus is generally most severe in the colon and typically lasts for 3 to 5 days. The administration of opioid analgesics to a patient after surgery may often contribute to bowel dysfunction, thereby delaying recovery of normal bowel function. Since a high proportion patients receive opioid analgesics, such as morphine or other narcotics, for pain relief after surgery, particularly major surgery, current post-surgical pain treatment may actually slow recovery of normal bowel function, resulting in a delay in hospital discharge and increasing the cost of medical care.

Post-surgical and post-partum ileus may also occur in the absence of exogenous opioid agonists. It would be of benefit to inhibit the natural activity of endogenous opioids during and/or after periods of biological stress, such as surgery and childbirth, so that ileus and related forms of bowel dysfunction can be prevented and/or treated. Currently, therapies for ileus have included functional stimulation of the intestinal tract, stool softeners, laxatives, lubricants, intravenous hydration, and nasogastric decompression. These prior art methods suffer from drawbacks, for example, as lacking specificity for post-surgical or post-partum ileus. And these prior art methods offer no means for prevention. If ileus could be prevented, hospital stays, recovery times, and medical costs would be significantly decreased, in addition to the benefit of minimizing patient discomfort. Thus, drugs that selectively act on opioid receptors in the gut would be ideal candidates for preventing and/or treating post-surgical and post-partum ileus. Of those, drugs that do not interfere with the effects of opioid analgesics in the CNS would be of special benefit in that they may be administered simultaneously for pain management with limited side effects.

Peripheral opioid antagonists that do not cross the blood-brain barrier into the CNS are known in the literature and have been tested in relation to their activity on the GI tract. In U.S. Pat. Nos. 5,250,542, 5,434,171, 5,159,081, and 5,270,328, peripherally selective piperidine-N-alkylcarboxylate opioid antagonists are described as being useful in the treatment of idiopathic constipation, irritable bowel syndrome and opioid-induced constipation. Also, U.S. Pat. No. 4,176,186 describes quaternary derivatives of noroxymorphone (i.e., methylnaltrexone) that are said to prevent or relieve the intestinal immobility side-effect of narcotic analgesics without reducing analgesic effectiveness. U.S. Pat. No. 5,972,954 describes, inter alia, the use of methylnaltrexone, enteric coated methylnaltrexone, or other quaternary derivatives of noroxymorphone for preventing and/or treating opioid-induced side effects associated with opioid administration.

General opioid antagonists, such as naloxone and naltrexone, have also been implicated as being useful in the treatment of GI tract dysmotility. For example, U.S. Pat. No. 4,987,126 and Kreek, M. J. Schaefer, R. A., Hahn, E. F., Fishman, J. Lancet 1983, 1(8319), 261 disclose naloxone and other morphinan-based opioid antagonists (i.e., naltrexone) for the treatment of idiopathic gastrointestinal dysmotility. In addition, naloxone has been shown to effectively treat non-opioid induced bowel obstruction, implying that the drug may act directly on the GI tract or in the brain (Schang, J. C., Devroede, G. Am. J. Gastroenerol. 1985, 80(6), 407). Furthermore, it has been implicated that naloxone may provide therapy for paralytic ileus (Mack, D. J. Fulton, J. D. Br. J. Surg. 1989, 76(10), 1101). However, it is well known that the activity of naloxone and related drugs is not limited to peripheral systems and may undesirably interfere with the analgesic effects of opioid narcotics.

Inasmuch as post-surgical and post-partum ileus are common illnesses that add to the cost of health care, there continues to be a need for specific and effective remedies. The majority of currently known opioid antagonist therapies are not peripherally selective and have the potential for undesirable side effects resulting from penetration into the CNS. Given the estimated 21 million inpatient surgeries and 26 million outpatient surgeries each year, and an estimate of 4.7 million patients experiencing post-surgical ileus, methods involving opioid antagonists that are not only specific for peripheral systems, but specific for the gut, are desirable for treating post-surgical and post-partum ileus.

There is still an unfulfilled need for compounds that may be used in methods to antagonize μ opioid receptors, particularly where the μ opioid receptor antagonist compounds may selectively target peripheral μ opioid receptors to ameliorate, inter alia, undesirable side effects associated with the chronic administration of exogenous opioids that are substantially mediated by μ opioid receptors. There is yet a further unfilled need for μ opioid receptor antagonist compounds to ameliorate undesirable symptoms or conditions where those undesirable symptoms or conditions are the result of surgical procedures, especially abdominal surgery. The present invention is directed to these, as well as other important ends.

### SUMMARY OF THE INVENTION

Accordingly, the present invention is directed, in part, to novel 3,4-disubstituted-4-(3-carbamoylphenyl)piperidinylpropanoic acid compounds. In particular, the present invention is directed to compounds of Formula I:

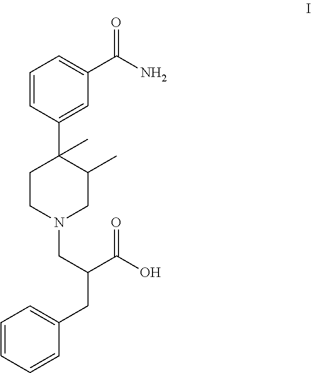

or salts thereof, preferably pharmaceutically acceptable salts.

In preferred embodiments, the present invention is directed to compounds of Formula IA:

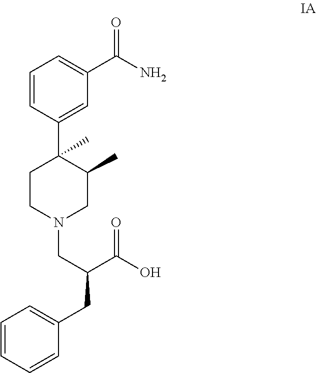

or salts thereof, preferably pharmaceutically acceptable salts.

In another embodiment, the invention is directed to pharmaceutical compositions comprising a pharmaceutically acceptable carrier and an effective amount of a compound of Formula I, preferably a compound of Formula IA, or pharmaceutically acceptable salts thereof.

In yet another embodiment, the invention is directed to methods for binding opioid receptors, including μ or κ opioid receptors or combinations thereof, preferably μ opioid receptors, where the methods comprise contacting the opioid receptors in vitro or in vivo with a compound of Formula I, preferably a compound of Formula IA.

In still another embodiment, the invention is directed to methods for binding opioid receptors, where the 3,4-disubstituted-4-(3-carbamoylphenyl)piperidinylpropanoic acid compounds exhibit activity toward opioid receptors, including μ or κ opioid receptors or combinations thereof, preferably μ opioid receptors, where the methods comprise contacting in vitro or in vivo the opioid receptors with a compound of Formula I, preferably a compound of Formula IA.

In some preferred embodiments, the invention is directed to methods for binding opioid receptors in a patient, where the methods comprise administering to the patient a compound of Formula I, preferably a compound of Formula IA, or pharmaceutically acceptable salts thereof. In certain preferred embodiments, the patient is in need of treatment of a condition, disease or undesirable side effect associated with surgical procedures, such as abdominal surgery, and/or one or more endogenous and/or exogenous opioids.

In certain preferred embodiments, the invention is directed to methods for treating gastrointestinal dysfunction in a patient, where the methods comprise administering to the patient a compound of Formula I, preferably a compound of Formula IA, or pharmaceutically acceptable salts thereof. Exemplary forms of gastrointestinal dysfunction include, for example, ileus, opioid bowel dysfunction and opioid-induced constipation.

In yet other preferred embodiments, the invention is directed to methods of treating pain comprising administering to a patient a composition comprising an effective amount of an opioid analgesic and an effective amount of a compound of Formula I, preferably a compound of Formula IA, or pharmaceutically acceptable salts thereof.

### BRIEF DESCRIPTION OF THE DRAWINGS

FIG. 1 is a graphical representation of experiments in mice on the antagonism of the anti-transit effect of morphine by a compound according to the invention and a compound of the prior art, as a function of time.

FIG. 2 is a graphical representation of experiments in rats of the total plasma concentration of a compound according to the present invention and a compound of the prior art, as a function of time, and the relative predictability of their comparative pharmacokinetics.

FIG. 3 is a graphical representation of experiments in dogs of the total plasma concentration of a compound according to the present invention and a compound of the prior art, as a function of time, and the relative predictability of their comparative pharmacokinetics.

FIG. 4 is a graphical representation of experiments in monkeys of the total plasma concentration of a compound according to the present invention and a compound of the prior art, as a function of time, and the relative predictability of their comparative pharmacokinetics.

### DETAILED DESCRIPTION OF ILLUSTRATIVE EMBODIMENTS

As employed above and throughout the disclosure, the following terms, unless otherwise indicated, shall be understood to have the following meanings.

“Stereoisomers” refers to compounds that have identical chemical constitution, but differ as regards the arrangement of the atoms or groups in space.

As used herein, the term “partial stereoisomers” refers to stereoisomers having two or more chiral centers wherein at least one of the chiral centers has defined stereochemistry (i.e., R or S) and at least one has undefined stereochemistry (i.e., R or S). When the term “partial stereoisomers thereof” is used herein, it refers to any compound within the described genus whose configuration at chiral centers with defined stereochemistry centers is maintained and the configuration of each undefined chiral center is independently selected from R or S. For example, if a stereoisomer has three chiral centers and the stereochemical configuration of the first center is defined as having “S” stereochemistry, the term “or partial stereoisomer thereof” refers to stereoisomers having SRR, SRS, SSR, or SSS configurations at the three chiral centers, and mixtures thereof.

Preferred embodiments of the invention involve compounds of Formula I in which the methyl substituents on the piperidine ring are in a “trans” orientation. The absolute stereochemistries of the carbon atoms in the piperidine ring to which the methyl groups are attached are also defined using the commonly employed “R” and “S” definitions (Orchin et al., The Vocabulary of Organic Chemistry, John Wiley and Sons, Inc., page 126, the disclosures of which are hereby incorporated herein by reference in their entireties). Preferred compounds of the present invention include those of Formula I and/or salts thereof, in which the configuration of both piperidine ring stereocenters bearing the methyl groups is “R”.

The present invention contemplates individual stereoisomers and/or combinations or mixtures of one or more stereoisomers and/or partial stereoisomers, as well as racemic mixtures. For example, compounds of Formula I have three stereocenters, denoted by the asterisks in the illustration below. Each of the stereocenters may have an R or S configuration. Thus, Formula I encompasses eight possible stereoisomers, each having one of the following stereochemical assignments: RRR, RRS, RSR, SRR, RSS, SRS, SSR, or SSS. Likewise, salts of compounds of Formula I may also have stereoisomeric structures with similar stereochemical assignments.

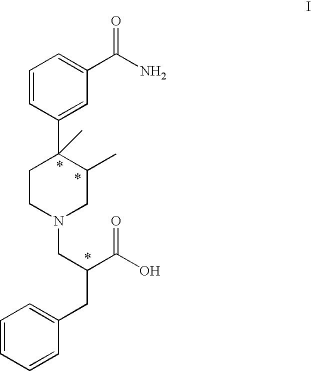

“Pharmaceutically acceptable” refers to those compounds, materials, compositions, salts and/or dosage forms which, within the scope of sound medical judgment, are suitable for administration to patients without excessive toxicity, irritation, allergic response, or other problems or complications commensurate with a reasonable benefit/risk ratio.

“Salts” refer to derivatives of the disclosed compounds wherein the parent compound is modified by making acid or base salts thereof, or wherein the parent compound is in its zwitterionic form. When contacted with an acid, for example, resulting in the protonation of an amine functionality, the compound becomes associated with an anion, i.e., the counterion of the acid. When contacted with a base, for example, resulting in the deprotonation of an acid functionality, the compound is associated with a cation, i.e., the counterion of the base. Examples of salts include, but are not limited to, mineral or organic acid salts of basic residues such as amines, alkali or organic base salts of acidic residues such as carboxylic acids, and the like. Suitable mineral or organic acids or bases that may be employed in preparing salts of the compounds of the invention would be readily apparent to one of ordinary skill in the art, once placed in possession of the present application.

In certain preferred embodiments, the salts are “pharmaceutically acceptable salts”, which include, for example, conventional salts derived from pharmaceutically acceptable acids or bases, as well as internal or zwitterionic salts. Such pharmaceutically acceptable salts include those derived from inorganic acids such as hydrochloric, hydrobromic, sulfuric, sulfamic, phosphoric or nitric acid and the like; and salts prepared from organic acids such as acetic, propionic, succinic, glycolic, stearic, lactic, malic, tartaric, citric, ascorbic, pamoic, maleic, hydroxymaleic, phenylacetic, aspartic, glutamic, benzoic, salicylic, sulfanilic, acetoxybenzoic, fumaric, toluenesulfonic, naphthyldisulfonic, methanesulfonic, ethane disulfonic, oxalic or isethionic acid, and the like. Pharmaceutically acceptable salts also include those derived from metal bases, including alkali metal bases, for example, alkali hydroxides such as sodium hydroxide, potassium hydroxide and lithium hydroxide in which the metal is a monovalent species, alkaline earth metal bases, for example, alkaline earth metal hydroxides such as magnesium hydroxide and calcium hydroxide in which the metal is a polyvalent species, basic amines such as, for example, N,N′-dibenzylethylenediamine, arginine, chloroprocaine, choline, diethanolamine, ethylenediamine, meglumine (N-methylglucamine) and procaine, ammonium bases or alkoxides.

Physiologically acceptable salts as described herein may be prepared by methods known in the art, for example, by dissolving the free amine bases with an excess of the acid in aqueous alcohol, or neutralizing a free carboxylic acid with a metal base, preferably an alkali metal base such as a hydroxide, a substituted or unsubstituted ammonium hydroxide, an alkoxide, or an amine. In addition, it is well known to ordinarily skilled artisans that in compounds containing, for example, both a basic nitrogen atom and an acidic group, the nitrogen atom and the acidic functionalities may exist in equilibrium with their zwitterionic form depending, for example, on the characteristics of the involved aqueous medium including, for example, its ionic strength, pH, temperature, salts involved when the aqueous medium is in the form of a buffer, and the like. These zwitterionic salts are, in essence, internal pharmaceutically acceptable salts, and are contemplated to be within the scope of the present invention.

The term “ammonium base”, as used herein, refers to ammonium hydroxide (NH₄OH), as well as substituted ammonium hydroxides, i.e., NR₄OH, where one, two, three or four of the R groups may be, independently, alkyl, cycloalkyl, alkenyl, aryl, aralkyl, heteroaryl, or heterocycloalkyl. Exemplary substituted ammonium hydroxides include, for example, tetraalkyl ammonium hydroxides, such as tetramethyl ammonium hydroxide.

The term “alkoxide”, as used herein, refers to the product from the reaction of an alkyl alcohol with a metal. Exemplary alkoxides include, for example, sodium ethoxide, potassium ethoxide and sodium t-butoxide.

Compounds described herein may be used or prepared in alternate forms. For example, many amino-containing compounds can be used or prepared as acid addition salts. Often such salts improve isolation and handling properties of the compound. The acid employed in forming acid addition salts is not generally limited. Pharmaceutically acceptable and pharmaceutically unacceptable acids may be used to prepare acid addition salts. For example, depending on the reagents, reaction conditions and the like, compounds as described herein can be used or prepared, for example, as their hydrochloride or tosylate salts. Similarly, compounds as described herein can be used or prepared, for example, as their oxalic acid or succinic acid salts, wherein one or both, preferably one, of the carboxylic acid groups in oxalic or succinic acid protonates the basic nitrogen atom in the compound of Formula I, preferably the compound of Formula IA.

Generally speaking, pharmaceutically unacceptable salts are not useful as medicaments in vivo. However, such salts may in certain cases demonstrate improved crystallinity and thus may be useful, for example, in the synthesis of compounds of Formula I, such as in connection with the formation, isolation and/or purification of compounds of Formula I and/or intermediates thereto. This may result, for example, in improved synthesis, purification or formulation by preparing and/or using compounds of the invention as salts that may not typically be considered to be pharmaceutically acceptable salts. These non-pharmaceutically acceptable salts may be prepared from acids or bases that are not typically considered to be pharmaceutically acceptable. Examples of such salts include, for example, acid addition salts prepared from trifluoroacetic acid, perchloric acid and tetrafluoroboric acid. Non-pharmaceutically acceptable salts may be employed in certain embodiments of the present invention including, for example, methods for the in vitro binding of opioid receptors. In addition, if desired, such non-pharmaceutically acceptable salts may be converted to pharmaceutically acceptable salts by using techniques well known to the ordinarily skilled artisan, for example, by exchange of the acid that is non-pharmaceutically acceptable, for example, trifluoroacetic, perchloric or tetrafluoroboric acid, with an acid that is pharmaceutically acceptable, for example, the pharmaceutically acceptable acids described above.

Acid addition salts of the present invention include, for example, about one or more equivalents of monovalent acid per mole of the compound of the invention, depending in part on the nature of the acid as well as the number of basic lone pairs of electrons available for protonation. Similarly, acid addition salts of the present invention include, for example, about one-half or more equivalents of a divalent acid (such as, for example, oxalic acid or succinic acid) or about one third or more equivalents of trivalent acid (such as, for example, citric acid) per mole of the compound of the invention, depending in part on the nature of the acid as well as the number of basic lone pairs of electrons available for protonation. Generally speaking, the number of acid equivalents may vary up to about the number of equivalents of basic lone pairs of electrons in the compounds described herein.

Salts of the present invention which are derived from metal bases or basic amines include, for example, about one or more equivalents of monovalent metal or amine per mole of the compound of the invention, depending in part on the nature of the base as well as the number of available acidic protons. Similarly, salts of the present invention include, for example, about one-half or more equivalents of a divalent base (such as, for example, magnesium hydroxide or calcium hydroxide). Generally speaking, the number of basic equivalents may vary up to about the number of equivalents of acidic protons in the compounds described herein.

As used herein, the term “hydrate” refers to a compound or salt as described herein which is associated with water in the molecular form, i.e., in which the H—OH bond is not split, and may be represented, for example, by the formula R.H₂O, where R is a compound as described herein. A given compound or salt may form more than one hydrate including, for example, monohydrates (R.H₂O) or polyhydrates (R.nH₂O wherein n is an integer>1) including, for example, dihydrates (R.2H₂O), trihydrates (R.3H₂O), and the like, or hemihydrates, such as, for example, R.n_{/2}H₂O, R.n_{/3}H₂O, R.n_{/4}H₂O and the like wherein n is an integer.

As used herein, the term “solvate” refers to a compound or salt as described herein which is associated with solvent in the molecular form, i.e., in which the solvent is coordinatively bound, and may be represented, for example, by the formula R.(solvent), where R is a compound as described herein. A given compound or salt may form more than one solvate including, for example, monosolvates (R.(solvent)) or polysolvates (R.n(solvent)) wherein n is an integer>1) including, for example, disolvates (R.2(solvent)), trisolvates (R.3(solvent)), and the like, or hemisolvates, such as, for example, R.n_{/2}(solvent), R.n_{/3}(solvent), R.n_{/4}(solvent) and the like wherein n is an integer. Solvents herein include mixed solvents, for example, methanol/water, and as such, the solvates may incorporate one or more solvents within the solvate.

As used herein, the term “acid salt hydrate” refers to a complex that may be formed through association of a compound having one or more base moieties with at least one compound having one or more acid moieties, the complex being further associated with water so as to form a hydrate.

“Side effect” refers to a consequence other than the one(s) for which an agent or measure is used, as the adverse effects produced by a drug, especially on a tissue or organ system other than the one sought to be benefited by its administration. In the case, for example, of opioids, the term “side effect” may refer to such conditions as, for example, constipation, nausea and/or vomiting, and respiratory depression.

“Effective amount” refers to an amount of a compound as described herein that may be therapeutically effective to treat the symptoms of one or more diseases, disorders or side effects. Such diseases, disorders and side effects include, but are not limited to, those pathological conditions associated with the administration of opioids (for example, in connection with the treatment of pain), wherein the treatment comprises, for example, affecting the disease, disorder and/or side effect by contacting cells, tissues or receptors with compounds of the present invention. Thus, for example, the term “effective amount”, when used in connection with opioids, for example, for the treatment of pain, refers to the treatment of the painful condition. The term “effective amount”, when used in connection with opioid antagonist compounds, refers to the treatment of, for example, side effects typically associated with opioids including, for example, such side effects as constipation, nausea and/or vomiting, as well as other side effects, discussed in further detail below. The term “effective amount,” when used in connection with compounds active against gastrointestinal dysfunction, refers to the treatment of symptoms, diseases, disorders, and conditions typically associated with gastrointestinal dysfunction including, for example, the alleviation and/or amelioration of ileus, opioid-induced bowel dysfunction and/or opioid-induced constipation.

“In combination with”, “combination therapy” and “combination products” refer, in certain embodiments, to the concurrent administration to a patient of one or more compounds or salts of the invention, in combination with one or more opioids.

The opioids may themselves further include one or more conventional anti-tussives, one or more compounds that may be designed to enhance the analgesic potency of the opioid and/or reduce analgesic tolerance development, and/or other therapeutic agents described herein. When administered in combination, each component may be administered at the same time or sequentially in any order at different points in time. Thus, each component may be administered separately but sufficiently closely in time so as to provide the desired therapeutic effect.

“Dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

“Patient” refers to animals, including mammals, preferably humans.

The terms “treat”, “treatment” or “treating”, as used herein, generally refer to palliative (e.g., therapeutic), preventative (e.g., prophylactic), inhibitory and/or curative treatment. Preferably, the terms “treat”, “treatment” and/or “treating” refer to palliative, inhibitory, and/or curative treatment, with palliative and inhibitory treatment being more preferred. In particularly preferred embodiments, the terms “treat”, “treatment” or “treating” refer to palliative treatment.

“Pain” refers to the perception or condition of unpleasant sensory or emotional experience, associated with actual or potential tissue damage or described in terms of such damage. “Pain” includes, but is not limited to, two broad categories of pain: acute and chronic pain. Buschmann, H.; Christoph, T; Friderichs, E.; Maul, C.; Sundermann, B; eds.; Analgesics, Wiley-VCH, Verlag GMbH & Co. KgaA, Weinheim; 2002; Jain, K. K., “A Guide to Drug Evaluation for Chronic Pain”; Emerging Drugs, 5(2), 241-257 (2000), the disclosures of which are hereby incorporated herein by reference in their entireties. Non-limiting examples of pain include, for example, nociceptive pain, inflammatory pain, visceral pain, somatic pain, neuralgias, neuropathic pain, AIDS pain, cancer pain, phantom pain, and psychogenic pain, and pain resulting from hyperalgesia, pain caused by rheumatoid arthritis, migraine, allodynia and the like.

The term “gastrointestinal dysfunction”, as used herein, refers collectively to maladies of the gastrointestinal system, particularly the stomach and small and large intestines. Non-limiting examples of gastrointestinal dysfunction include, for example, diarrhea, nausea, emesis, post-operative emesis, opioid-induced emesis, irritable bowel syndrome, opioid-bowel dysfunction, opioid induced constipation, ileus, including post-operative ileus, post-partum ileus and opioid-induced ileus, colitis, decreased gastric motility, decreased gastric emptying, inhibition of small intestinal propulsion, inhibition of large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, distension, abdominal or epigastric pain and discomfort, non-ulcerogenic dyspepsia, gastritis, constipation, or delayed absorption of orally administered medications or nutritive substances.

The present invention is directed, in part, to substituted piperidinylpropanoic acid compounds that preferably bind and/or interact with opioid receptors. Embodiments are provided in which the compounds of the invention are 3,4-disubstituted-4-(3-carbamoyl-phenyl)piperidinylpropanoic acid compounds. In preferred form, compounds described herein may be antagonists of opioid receptors, particularly μ opioid receptors, with relatively diminished antagonist activity for κ and δ opioid receptors.

The 3,4-disubstituted-4-(3-carbamoyl-phenyl)piperidinylpropanoic acid compounds and salts thereof of the present invention demonstrate a surprisingly and unexpectedly advantageous profile of biological activities relative to profiles of biological activities of prior art compounds. In this regard, due to their desirable affinity for opioid receptors, especially μ opioid receptors, compounds and salts thereof as described herein may be useful, for example, in methods for binding such opioid receptors. Accordingly, the present compounds and pharmaceutically acceptable salts thereof may be useful in treating diseases or disorders that may be associated with and/or modulated by opioid receptors. In preferred embodiments, the present compounds and pharmaceutically acceptable salts thereof may be employed in methods for the treatment of gastrointestinal dysfunction that may be caused by surgical procedures, particularly abdominal surgery such as, for example, ileus, as well as in the treatment of diseases or disorders associated with opioids, particularly side effects associated with opioid administration, including constipation, nausea and vomiting, as well as opioid induced bowel dysfunction. Compounds of the present invention may be potent and selective antagonists of μ opioid receptors, especially μ opioid receptors expressed in the periphery, and may have highly desirable potencies as antagonists of μ opioid receptors. In addition, compounds of the present invention may demonstrate highly beneficial increases in in vivo oral bioavailability resulting in more predictable systemic exposure, and reduced variability in their pharmacokinetic behavior as compared to prior art compounds. This highly desirable profile of biological activities and pharmacokinetic properties in compounds of the present invention as compared to prior art compounds is surprising and unexpected.

In certain preferred embodiments, the compounds, pharmaceutical compositions and methods of the present invention may involve a peripheral opioid antagonist compound. The term “peripheral” designates that the compound acts primarily on physiological systems and components external to the central nervous system (CNS). In preferred form, the peripheral opioid antagonist compounds employed in the methods of the present invention exhibit high levels of activity with respect to peripheral tissue, such as, gastrointestinal tissue, while exhibiting reduced, and preferably substantially no, CNS activity at therapeutically relevant doses. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably non-detectible, de minimus, or even 0% of the pharmacological activity of the compounds employed in the present methods is exhibited in the CNS.

Furthermore, in embodiments of the invention where the compound is administered to antagonize the peripheral side effects of an opioid, it is generally preferred that the compound does not substantially cross the blood-brain barrier and thereby decrease the beneficial activities of opioids in the central nervous system. The phrase “does not substantially cross,” as used herein, means that less than about 20% by weight of the compound employed in the present methods crosses the blood-brain barrier, preferably less than about 15% by weight, more preferably less than about 10% by weight, even more preferably less than about 5% by weight and still more preferably a non-detectible, de minimus, or even 0% by weight of the compound crosses the blood-brain barrier at therapeutically relevant doses. Selected compounds can be evaluated for CNS penetration by determining plasma and brain levels following i.v., oral, subcutaneous or intraperitoneal administration.

Accordingly, in one embodiment, the present invention provides a compound of the following Formula I:

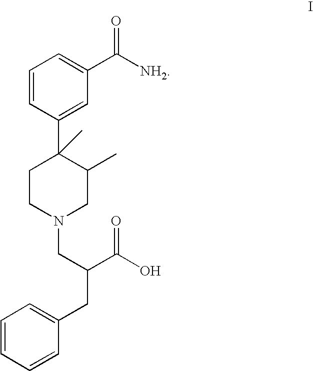

In certain preferred embodiments, the compound of Formula I may be in non-salt form, i.e., the compound is not contacted with an acid or base and is not associated with any cations or anions, and is not in zwitterionic form. In certain other preferred embodiments, the compound of Formula I may be in the form of a salt, preferably a pharmaceutically acceptable salt. Exemplary salts include, for example,

(a) carboxylate salts as represented, for example, by Formula I-S-1:

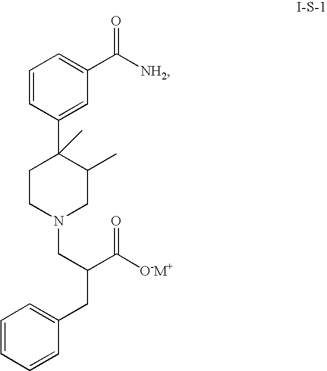

where M⁺ is a monovalent or polyvalent cation of a base, preferably a monovalent cation, preferably a pharmaceutically acceptable base, including alkali metal bases;

(b) zwitterionic or internal salts as represented, for example, by Formula I-S-2:

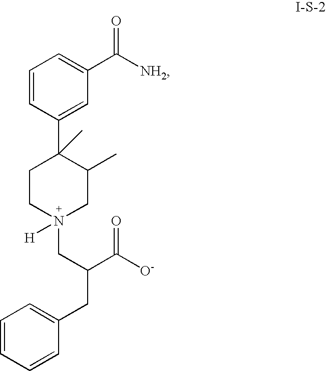

(c) or acid addition salts as represented, for example, by Formula I-S-3:

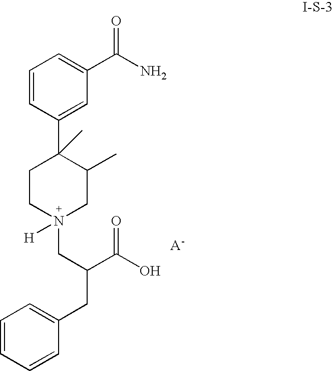

where A⁻ is a monovalent or polyvalent anion of an acid, preferably a monovalent anion, preferably a pharmaceutically acceptable acid, including inorganic or organic acids.

In certain particularly preferred embodiments, the compound of Formula I has the following Formula IA:

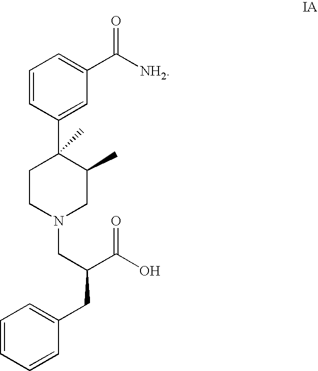

In certain particularly preferred embodiments, the compound of Formula IA is in the form of one or more salts, preferably pharmaceutically acceptable salts, including

(a′) carboxylate salts of Formula IA-S-1:

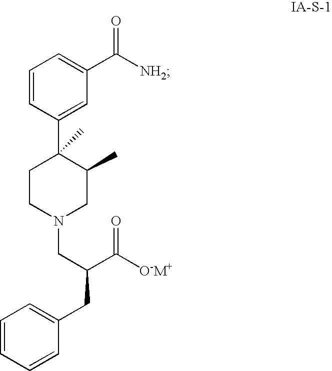

where M⁺ is a monovalent or polyvalent cation derived from a base, preferably a monovalent cation, preferably a pharmaceutically acceptable base, including alkali metal bases,

(b′) zwitterionic salts of Formula IA-S-2:

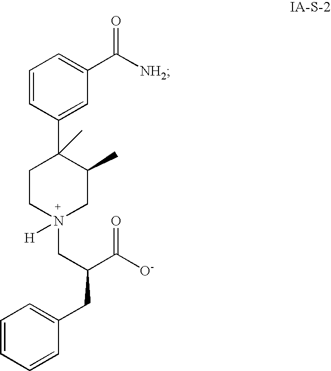

and

(c′) acid addition salts of Formula IA-S-3.

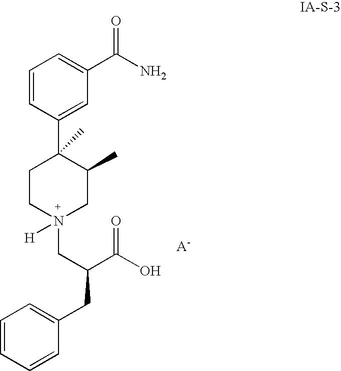

where A⁻ is a monovalent or polyvalent anion derived from an acid, preferably a pharmaceutically acceptable acid, including inorganic or organic acids.

In accordance with embodiments of the present invention such as, for example, pharmaceutical compositions, the pharmaceutically active agent included therein may be the compound of Formula I, a pharmaceutically acceptable salt of Formula I-S-1, a pharmaceutically acceptable salt of Formula I-S-2 or a pharmaceutically acceptable salt of Formula I-S-3, or various combinations of the compound of Formula I (and/or specific stereoisomers thereof) and/or one or more pharmaceutically acceptable salts of Formulas I-S-1, I—S-2 and I—S-3 (and/or specific stereoisomers thereof).

Salts of Formula I-S-1 are carboxylate salts. The term “carboxylate salt”, as used herein, refers to a salt derived from a compound, for example, a compound of Formula I, that contains a carboxylic acid (COOH) group in which the proton has been removed to provide a carboxylate (COO⁻) group. Typically, proton removal may be carried out by contacting the carboxylic acid compound with a base, including a pharmaceutically acceptable base, for example, an alkali metal base, an amine base, an ammonium base or an alkoxide base, as described above. Compounds of Formula I in which the carboxylic acid group has been converted to a carboxylate group may be preferred salts in accordance with certain embodiments of the invention. Other bases that may be employed in preparing the carboxylate salts of the present invention would be readily apparent to one of ordinary skill in the art, once armed with the teachings in the present application.

In embodiments involving pharmaceutically acceptable salts of, for example, Formula I-S-1, the cation M⁺ may be, for example, a metal cation, including monovalent metal cations such as a sodium, potassium or lithium cation, with sodium and lithium cations being preferred, and sodium cations being more preferred. In alternate embodiments, the metal cation may be a polyvalent cation, for example, a divalent cation such as a magnesium or calcium cation. In still other alternate embodiments, the cation may be, for example, an ammonium ion.

In embodiments involving salts of, for example, Formulas I-S-3, the anion A⁻ may correspond to the counterion of a mineral or organic acid after removal of one or more protons and may be monovalent or polyvalent (such as for example, di- or trivalent). Accordingly, in the case of pharmaceutically acceptable acids such as hydrochloric, hydrobromic, sulfuric, sulfamic, phosphoric, nitric, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, acetoxybenzoic, fumaric, toluenesulfonic, methanesulfonic, ethane disulfonic, oxalic, and isethionic acid, the anion A⁻ may be, for example, chloride (Cl⁻), bromide (Br⁻), sulfate (SO₄⁻²), hydrogensulfate (HSO₄⁻), sulfamate (SO₃.NH₂⁻), phosphate (PO₄⁻³), dihydrogenphosphate (H₂PO₄⁻), hydrogenphosphate (HPO₄⁻²), nitrate (NO₃⁻), acetate (CH₃CO₂⁻), propionate (CH₃CH₂CO₂⁻), succinate (C₂H₄(CO₂⁻)₂ or C₂H₄(CO₂H)(CO₂⁻)), 3-carboxypropanoate (C₂H₄(COOH)(CO₂⁻), glycolate (HOCH₂CO₂⁻), stearate (CH₃(CH₂)₁₆CO₂⁻), lactate (CH₃CH(OH)CO₂⁻), malate (CH(OH)(CO₂⁻)CH₂CO₂⁻ or CH(OH)(CO₂H)CH₂CO₂⁻), 3-carboxy-2-hydroxypropanoate (CH(OH)(CO₂⁻)CH₂CO₂H), 3-carboxy-3-hydroxypropanoate (CH(OH)(CO₂H)CH₂CO₂⁻), tartrate (⁻O₂CCH(OH)CH(OH)CO₂⁻ or HO₂CCH(OH)CH(OH)CO₂⁻), 3-carboxy-2,3-dihydroxypropanoate (HO₂CCH(OH)CH(OH)CO₂⁻), citrate (C₃H₄(OH)(CO₂H)₂(CO₂⁻), C₃H₄(OH)(CO₂H)(CO₂⁻)₂ or C₃H₄(OH)(CO₂⁻)₃), 2-(carboxymethyl)-2-hydroxysuccinate (C₃H₄(OH)(COOH)(CO₂⁻)₂ or C₃H₄(OH)(CO₂H)₂(CO₂⁻)), 3-carboxy-3-hydroxypentanedioate (C₃H₄(OH)(CO₂H)(CO₂⁻)₂) or C₃H₄(OH)(CO₂H)₂(CO₂⁻)), 3-carboxy-2-(carboxymethyl)-2-hydroxypropanoate (C₃H₄(OH)(COOH)₂(CO₂⁻)), 3,4-dicarboxy-3-hydroxybutanoate (C₃H₄(OH)(COOH)₂(CO₂⁻)), ascorbate, pamoate (C₂₃H₁₄O₆⁻²), maleate (C₂H₂(CO₂⁻)₂ or HO₂CC₂H₂CO₂⁻), (Z)-3-carboxyacrylate ((C₂H₂(COOH)(CO₂⁻)), phenylacetate (C₆H₅CH₂CO₂⁻), 2-aminosuccinate (H₂NCH(CO₂⁻)CH₂CO₂⁻), aspartate (H₂NCH(CO₂⁻)CH₂CO₂H), 3-amino-3-carboxypropanoate (H₂NCH(CO₂H)CH₂CO₂⁻), 2-aminopentanedioate (H₂NCH(CO₂⁻)(CH₂)₂CO₂⁻), glutamate (H₂NCH(CO₂⁻)(CH₂)₂CO₂H), 4-amino-4-carboxybutanoate (H₂NCH(CO₂H)(CH₂)₂CO₂⁻), benzoate (C₆H₅CO₂⁻), salicylate (C₆H₄(OH)CO₂⁻), sulfanilate (H₂NC₆H₄SO₃⁻), acetoxybenzoate (C₆H₄(—O—C(═O)CH₃)CO₂⁻), fumarate (C₂H₂(CO₂⁻)₂ or C₂H₂(CO₂H)CO₂⁻), (E)-3-carboxyacrylate (C₂H₂(COOH)CO₂⁻)), toluenesulfonate (C₆H₄(CH₃)SO₃⁻), naphthyldisulfonate (C₁₀H₆)(SO₃⁻)₂), sulfonaphthalene-sulfonate (C₁₀H₆)(SO₃H)(SO₃⁻), methanesulfonate (CH₃SO₃⁻) ethane disulfonate (^{-O}₃S(CH₂)₂SO₃⁻), sulfoethane-sulfonate (HO₃S(CH₂)₂SO₃⁻), oxalate ((CO₂⁻)₂), carboxyformate HOOC—(CO₂⁻), or isethionate (CH₂OHCH₂SO₃⁻) ions. In certain more preferred embodiments, the pharmaceutically acceptable salts include those derived from oxalic or succinic acid. In the case of non-pharmaceutically acceptable acids, such as trifluoroacetic, perchloric and tetrafluoroboric acid, the anion A⁻ may be, for example, trifluoroacetate (CF₃CO₂⁻), perchlorate (ClO₄⁻) and tetrafluoroborate (BF₄⁻) ions. Other acids, including non-pharmaceutically acceptable acids and pharmaceutically acceptable acids, that may be employed in preparing the acid addition salts of the present invention would be readily apparent to one of ordinary skill in the art, once armed with the teachings in the present application.

Compounds of the invention, such as a compound of Formula I and salts thereof, also include other forms, such as their stereoisomers (except where specifically indicated), prodrugs, hydrates, solvates, acid salt hydrates, or any isomorphic crystalline forms thereof.

Compounds employed in the methods and compositions of the present invention may exist in prodrug form. As used herein, “prodrug” is intended to include any covalently bonded carriers which release the active parent drug, for example, the compound of Formula I, or other formulas or compounds employed in the present methods and compositions in vivo when such prodrug is administered to a mammalian subject. The term “prodrug” also includes compounds which may be specifically designed to maximize the amount of active species that reaches the desired site of reaction and which themselves may be inactive or minimally active for the activity desired, but through biotransformation are converted into biologically active metabolites. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example a compound of Formula I, may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

Compounds employed in the methods and compositions of the present invention may also be substituted with or enriched in heavier isotopes such as deuterium, i.e., ²H. Such deuterated compounds may afford certain therapeutic advantages resulting from greater metabolic stability, for example increased in vivo half-life or reduced dosage requirements and, hence, may be preferred in some circumstances. Isotopically labeled compounds of Formula I can generally be prepared by carrying out the procedures disclosed in the Schemes and/or in the Examples below, by substituting a readily available isotopically labeled reagent for a non-isotopically labeled reagent. The degree of enrichment may vary, and is preferably from greater than the natural abundance of deuterium (i.e., 0.015%) such as for example, from about 0.5% to 100% (and all combinations and subcombinations of ranges of enrichment and specific enrichment values therein).

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As discussed in detail above, compounds of the present invention may contain one or more asymmetrically substituted carbon atoms, and may be isolated in optically active or racemic forms. Thus, all chiral, diastereomeric, and racemic forms and all geometric isomeric forms of any given structure are intended, unless the specific stereochemistry or isomeric form is specifically indicated. Techniques for isolating and preparing such optically active forms are well known in the art. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include benzyloxycarbonyl and tert-butyloxycarbonyl groups. Other preferred protecting groups that may be employed in accordance with the present invention may be described in Greene, T. W. and Wuts, P. G. M., Protective Groups in Organic Synthesis 2d. Ed., Wiley & Sons, 1991; or Kocienski, P. J., Protecting Groups, 3d. ed., Georg Thieme Verlag: Stuttgart, 2005, the disclosures of each of which are hereby incorporated herein by reference, in their entireties.

While not intending to be bound by any theory or theories of operation, it is contemplated that opioid side effects, such as constipation, vomiting and/or nausea, may result from undesirable interaction of an opioid with peripheral opioid receptors, in particular peripheral μ opioid receptors. According to one aspect of the present invention, administration of the compounds of the invention, such as a compound of Formula I or a pharmaceutically acceptable salt thereof, preferably a compound of Formula IA or pharmaceutically acceptable salt thereof, may block interaction of the opioid with peripheral receptors, thereby treating one or more side effects, while preferably not interfering with therapeutic effects of the opioid in the CNS.

In accordance with certain embodiments of the present invention, there are provided methods which comprise administering to a patient the compounds of the invention, such as a compound of Formula I, preferably the compound of Formula IA, or pharmaceutically acceptable salts thereof, for example, a pharmaceutically acceptable salt of Formula I-S-1, I-S-2 and/or I-S-3, alone or in combination with an opioid compound. A wide variety of opioids are available which may be suitable for use in such methods and compositions. Generally speaking, it is only necessary that the opioid provide the desired effect (for example, pain alleviation), and be capable of being incorporated into the present compositions and methods (discussed in detail below). In preferred embodiments, the present methods and compositions may involve an opioid which is selected from alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, loperamide, meperidine, meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, a diastereoisomer thereof, a pharmaceutically acceptable salt thereof, a complex thereof, or a mixture thereof; more preferably from alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil and/or tramadol. More preferably, the opioid is selected from morphine, codeine, oxycodone, hydrocodone, dihydrocodeine, propoxyphene, fentanyl and/or tramadol.

The opioid component of the present compositions may further include one or more other active ingredients that may be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosure of which is hereby incorporated herein by reference, in its entirety.

In addition, the compositions or opioid component may further include one or more compounds that may be designed to enhance the analgesic potency of the opioid and/or to reduce analgesic tolerance development. Such compounds include, for example, dextromethorphan or other NMDA antagonists (Mao, M. J. et al., Pain 1996, 67, 361), L-364,718 and other CCK antagonists (Dourish, C. T. et al., Eur J Pharmacol 1988, 147, 469), NOS inhibitors (Bhargava, H. N. et al., Neuropeptides 1996, 30, 219), PKC inhibitors (Bilsky, E. J. et al., J Pharmacol Exp Ther 1996, 277, 484), and dynorphin antagonists or antisera (Nichols, M. L. et al., Pain 1997, 69, 317). The disclosures of each of the foregoing documents are hereby incorporated herein by reference, in their entireties.

Other opioids, optional conventional opioid components, and optional compounds for enhancing the analgesic potency of the opioid and/or for reducing analgesic tolerance development that may be employed in the methods and compositions of the present invention, in addition to those exemplified above, would be readily apparent to one of ordinary skill in the art, once armed with the teachings of the present disclosure.

Another embodiment of the invention provides pharmaceutical compositions comprising a pharmaceutically acceptable carrier and an effective amount of a compound of the invention, such as a compound of Formula I or pharmaceutically acceptable salts thereof, preferably a compound of Formula IA or pharmaceutically acceptable salts thereof, for example, a pharmaceutically acceptable salt of Formula IA-S-1, IA-S-2 and/or IA-S-3.

Yet another embodiment of the invention provides methods for treating gastrointestinal dysfunction comprising administering to a patient in need of such treatment an effective amount of a compound of the invention, such as a compound of Formula I or pharmaceutically acceptable salts thereof, preferably a compound of Formula IA or pharmaceutically acceptable salts thereof, for example, a pharmaceutically acceptable salt of Formula IA-S-1, IA-S-2 and/or IA-S-3.

Preferred embodiments of the invention provide methods for treating ileus comprising administering to a patient in need of such treatment an effective amount of a compound of the invention, such as a compound of Formula I or pharmaceutically acceptable salts thereof, preferably a compound of Formula IA or pharmaceutically acceptable salts thereof, for example, a pharmaceutically acceptable salt of Formula IA-S-1, IA-S-2 and/or IA-S-3.

Another embodiment of the invention provides methods for treating one or more side effects associated with an opioid comprising administering to a patient an effective amount of compound of the invention, such as a compound of Formula I or pharmaceutically acceptable salts thereof, preferably a compound of Formula IA or pharmaceutically acceptable salts thereof, for example, a pharmaceutically acceptable salt of Formula IA-S-1, IA-S-2 and/or IA-S-3.

Although the compounds of the present invention may be administered as the pure chemicals, it is preferable to present the active ingredient as a pharmaceutical composition. The invention thus further provides a pharmaceutical composition comprising a compound of Formula I or pharmaceutically acceptable salt thereof, together with one or more pharmaceutically acceptable carriers therefor and, optionally, other therapeutic and/or prophylactic ingredients. The carrier(s) must be acceptable in the sense of being compatible with the other ingredients of the composition and not deleterious to the recipient thereof.

The compounds of the invention may be administered in an effective amount by any of the conventional techniques well-established in the medical field. Compounds employed in the methods of the present invention including, for example, one or more opioids and the compounds of the invention, such as a compound of Formula I or pharmaceutically acceptable salts thereof, may be administered by any means that results in the contact of the active agents with the agents' site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The compounds may be administered alone or may be combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety The relative proportions of active ingredient and carrier may be determined, for example, by the solubility and chemical nature of the compounds, chosen route of administration and standard pharmaceutical practice.

Compounds as described herein may be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, and rectal; nasal inhalation via insufflations and aerosols.

The active compound(s) may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations according to the present invention may be prepared so that an oral dosage unit form contains from about 0.1 to about 1000 mg of active compound, and all combinations and subcombinations of ranges and specific amounts of active compound therein.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and/or by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and/or freeze drying techniques that yield a powder of the active ingredient, plus any additional desired ingredient from the previously sterile-filtered solution thereof.

The dosage of the compounds of the invention may vary depending upon various factors such as, for example, the pharmacodynamic characteristics of the particular agent and its mode and route of administration, the age, health and weight of the recipient, the nature and extent of the symptoms, the kind of concurrent treatment, the frequency of treatment, and the effect desired. Generally, small dosages may be used initially and, if necessary, increased by small increments until the desired effect under the circumstances is reached. Generally speaking, oral administration may require higher dosages.

Although the proper dosage of the compounds of this invention will be readily ascertainable by one skilled in the art, once armed with the present disclosure, typically a dosage of the compound of the invention, preferably a compound of Formula I and/or pharmaceutically acceptable salts thereof, for example, a pharmaceutically acceptable salt of Formula I-S-1, I-S-2 and/or I-S-3, may range from about 0.001 to about 1000 milligrams, and all combinations and subcombinations of ranges and specific dosage amounts therein. Preferably, the dosage may be about 0.01 to about 100 milligrams of the compound or pharmaceutically acceptable salt of the invention, with from about 0.01 to about 10 milligrams being more preferred.

Combination products of this invention, such as pharmaceutical compositions comprising opioid(s) in combination with a compound of the invention, such as a compound of Formula I or pharmaceutically acceptable salts thereof, for example, pharmaceutically acceptable salts of Formula I-S-1, I-S-2 and/or I-S-3, may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the opioid compound(s) and compound of the invention or pharmaceutically acceptable salt thereof may be administered at the same time (that is, together), or in any order. When not administered at the same time, preferably the administration of an opioid and a compound of the invention or pharmaceutically acceptable salt thereof occurs less than about 8 hours apart, more preferably less than about 4 hours apart, more preferably less than about 2 hours apart, more preferably less than about one hour apart, more preferably less than about 30 minutes apart, even more preferably less than about 15 minutes apart, and still more preferably less than about 5 minutes apart. Preferably, administration of the combination products of the invention is oral, although other routes of administration, as described above, are contemplated to be within the scope of the present invention. Although it is preferable that the opioid(s) and compound of the invention or pharmaceutically acceptable salt thereof are both administered in the same fashion (that is, for example, both orally), if desired, they may each be administered in different fashions (that is, for example, a first component of the combination product may be administered orally, and a second component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Although the proper dosage of the combination products of this invention will be readily ascertainable by one skilled in the art, once armed with the present disclosure, by way of general guidance, where an opioid compound is combined with the compound of the invention, such as a compound of Formula I or pharmaceutically acceptable salt thereof, typically a dosage may range from about 0.01 to about 100 milligrams of the opioid (and all combinations and subcombinations of ranges and specific dosage amounts therein) and about 0.001 to about 100 milligrams of a compound of the invention, such as a compound of Formula I or pharmaceutically acceptable salt thereof (and all combinations and subcombinations of ranges and specific dosage amounts therein). Preferably, a dosage may be about 0.1 to about 10 milligrams of the opioid and about 0.01 to about 10 milligrams of a compound of the invention or pharmaceutically acceptable salt thereof. With regard to a typical dosage form of this type of combination product, such as a tablet, the opioid compounds (e.g., morphine) generally may be present in an amount of about 15 to about 200 milligrams (and all combinations and subcombinations of ranges and specific amounts therein), and a compound of the invention or pharmaceutically acceptable salt thereof may generally be present in an amount of about 0.1 to about 4 milligrams (and all combinations and subcombinations of ranges and specific amounts therein).

When provided as a single dosage form, the potential exists for a chemical interaction between the combined active ingredients (for example, an opioid and a compound of the invention or pharmaceutically acceptable salt thereof. For this reason, the preferred dosage forms of the present combination products are formulated such that although the active ingredients are combined in a single dosage form, the physical contact between the active ingredients is minimized (i.e., reduced).

In order to minimize contact, one embodiment of this invention where the product is orally administered provides for a combination product wherein one active ingredient is enteric-coated. By enteric-coating one or more of the active ingredients, it is possible not only to minimize the contact between the combined active ingredients, but it is also possible to control the release of one of these components in the gastrointestinal tract such that one of these components is not released in the stomach but rather is released in the intestines.

Another embodiment of this invention where oral administration is desired provides for a combination product wherein one or more of the active ingredients is coated with a sustained-release material that effects a sustained-release throughout the gastrointestinal tract and also serves to minimize physical contact between the combined active ingredients. Furthermore, the sustained-released component(s) can be additionally enteric-coated such that the release of this component occurs only in the intestine. Still another approach would involve the formulation of a combination product in which one component is coated with a sustained and/or enteric release polymer, and another component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of combination products of the present invention wherein one active ingredient is enteric-coated can be in the form of tablets such that the enteric-coated component and the other active ingredient are blended together and then compressed into a tablet or such that the enteric-coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric-coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

Pharmaceutical kits useful in, for example, the treatment of pain, which comprise a therapeutically effective amount of an opioid along with a therapeutically effective amount of a compound of the invention or pharmaceutically acceptable salt thereof, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL™ two-part container (available from Abbott Labs, Chicago, Ill.), as desired. The opioid compound and a compound of the invention or pharmaceutically acceptable salt thereof may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

It will be further appreciated that the amount of the compound, or an active salt or derivative thereof, required for use in treatment will vary not only with the particular compound, salt or derivative selected but also with the route of administration, the nature of the condition being treated and the age and condition of the patient, and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The dose may also be provided by controlled release of the compound, by techniques well known to those in the art, for example, as either as a mono therapy or combination therapy with a narcotic.

Compounds of the present invention may be used in methods to bind opioid receptors in vitro or in vivo, including μ, δ and κ opioid receptors, particularly μ opioid receptors. Such binding may be accomplished by contacting the receptor in vitro or in vivo with an effective amount of a compound of the invention. Preferably, the contacting step is conducted in an aqueous medium, preferably at physiologically relevant ionic strength, pH, and the like. Methods of in vitro binding may involve, for example, pharmaceutically acceptable salts or non-pharmaceutically acceptable salts, and may be used, for example, in assays to evaluate the binding affinities of compounds of the invention for opioid receptors, in assays to evaluate the binding affinities of other compounds for opioid receptors in which the present compounds may be used as an assay standard, and the like.

In certain preferred embodiments, compounds of the present invention bind μ, δ, or κ opioid receptors or combinations thereof, particularly μ opioid receptors. The opioid receptors may be located in the central nervous system or located peripherally to the central nervous system or in both locations.

In preferred embodiments of the methods of the invention, compounds as described herein antagonize the activity of opioid receptors. In certain preferred embodiments, the compounds treat a condition or disease caused by an opioid (either endogenous or exogenous). In certain embodiments of the present methods, particularly where the opioid is exogenous, compounds of the invention preferably do not substantially cross the blood-brain barrier.

Compounds of the present invention may be used in methods to antagonize μ, δ or κ opioid receptors, or any combination thereof, especially μ opioid receptors, particularly where undesirable symptoms or conditions are side effects of administering exogenous opioids. Furthermore, the compounds of the invention may be used to treat patients having disease states that are ameliorated by binding opioid receptors or in any treatment wherein temporary suppression of μ, δ or κ opioid receptors, or any combination thereof, particularly μ opioid receptors, may be desired. Compounds of the invention may also be used to antagonize the μ opioid receptor without significantly antagonizing δ and/or κ opioid receptors.

The present methods may be employed in the treatment, including regulation and/or reversal of pruritus (itching), increased biliary tone, increased biliary colic, urinary retention, ileus, emesis, rapid opioid peripheral detoxification, potentiation of opioid analgesia (especially at ultra-low and low doses), opioid tolerance and physical dependence (especially at ultra-low and low doses), the immune system and cancers associated with binding of the opioid receptors; and regulation of blood pressure. As used herein, the term “low dose” refers to a dosage level from about 100 to about 1000 micrograms. As used herein, the term “ultra-low dose” refers to a dosage level from about 10 to about 100 micrograms.

In certain preferred embodiments, the compounds of the invention may be used in methods for treating gastrointestinal dysfunction, including, but not limited to, irritable bowel syndrome, opioid-bowel dysfunction, colitis, post-operative and opioid-induced emesis (nausea and vomiting), decreased gastric motility and emptying, inhibition of small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, and delayed absorption of orally administered medications or nutritive substances. In certain preferred embodiments, one or more of the foregoing symptoms may occur, at least in part, as a result of the administration of opioid analgesics.

In certain particularly preferred embodiments, the compounds of the invention may be used in methods for treating ileus, particularly post-operative ileus, post-partum ileus and/or opioid-induced ileus.

In other particularly preferred embodiments, the compounds of the invention may be used in methods for treating opioid bowel dysfunction.

Also in particularly preferred embodiments, the compounds of the invention may be used in methods for treating opioid-induced constipation.

In other preferred embodiments, the compounds of the invention may be used in combination with an effective amount of an opioid to treat pain. In these embodiments, the compounds of the invention preferably reduce peripheral opioid side effects, including for example gastrointestinal dysfunction that may be associated with the administration of the opioid.

In embodiments involving the administration of at least one opioid, the compounds of the invention may be administered before, during or after administering the opioid.

The 3,4-dimethyl-4-(3-carbamoylphenyl)piperidinylpropanoic acid compounds according to the present invention may be synthesized employing methods described, for example, in U.S. Pat. Nos. 5,250,542, 5,434,171, 5,159,081, and 5,270,328, the disclosures of which are hereby incorporated herein by reference, in their entireties. The optically active (+)-4(R)-(3-hydroxyphenyl)-3(R),4-dimethyl-1-piperidine that may be employed as a starting material in the synthesis of the present compounds may be prepared by the general procedure described in J. Org. Chem., 1991, 56, 1660-1663, U.S. Pat. No. 4,115,400 and U.S. Pat. No. 4,891,379, the disclosures of which are hereby incorporated herein by reference in their entireties.

### EXAMPLES

The present invention is further described in the following examples. A series of N-substituted (+)-4(R)-(3-substituted phenyl)-3(R),4-dimethyl-1-piperidinylpropanoic acid compounds were prepared according to procedures outlined in Schemes 1 to 7. Schemes 1 to 5 and Examples 1 to 6 describe the preparation of a compound and salts of the invention. Schemes 6 and 7 and comparative Examples C-1 and C-2 describe the preparation of compounds of the prior art. Comparative Example C-3 describes the preparation of an alternative salt form of a prior art compound. All of the examples are actual examples. These examples are for illustrative purposes only, and are not to be construed as limiting the appended claims.

Materials: all chemicals were reagent grade and used without further purification. Analytical thin-layer chromatography (TLC) was performed on silica gel glass plates (250 microns) from Analtech and visualized by UV irradiation and iodine. Chromatography was conducted with silica gel (200-400 mesh, 60 Å, Aldrich). Chromatographic elution solvent systems are reported as volume:volume ratios. LC-MS data were obtained using a LC Thermo Finnigan Surveyor-MS Thermo Finnigan AQA in either positive mode or negative mode. Solvent A: 10 mM ammonium acetate, pH 4.5; solvent B: acetonitrile; solvent C: methanol; solvent D: water; column Waters Xterra C18 MS 2.0×50 mm, detector: PDA λ is 220-300 nM. Gradient program (positive mode): t=0.00, 600 μL/min, 99% A-1% B; t=0.30, 600 μL/min, 99% A-1% B; t=5.00, 600 μL/min, 1% A-99% B; t=5.30, 600 μL/min, 1% A-99% B. Gradient program (negative mode): t=0.00, 600 μL/min, 9% A-1% B-90% D; t=0.30, 600 μL/min, 9% A-1% B-90% D; t=5.00, 600 μL/min, 99% B-1% D; t=5.30, 600 μL/min, 99% B-1% D.

### Example 1

### Preparation of (S)-2-benzyl-3-((3R,4R)-4-(3-carbamoylphenyl)-3,4-dimethylpiperidin-1-yl)propanoic acid, lithium salt (4a)

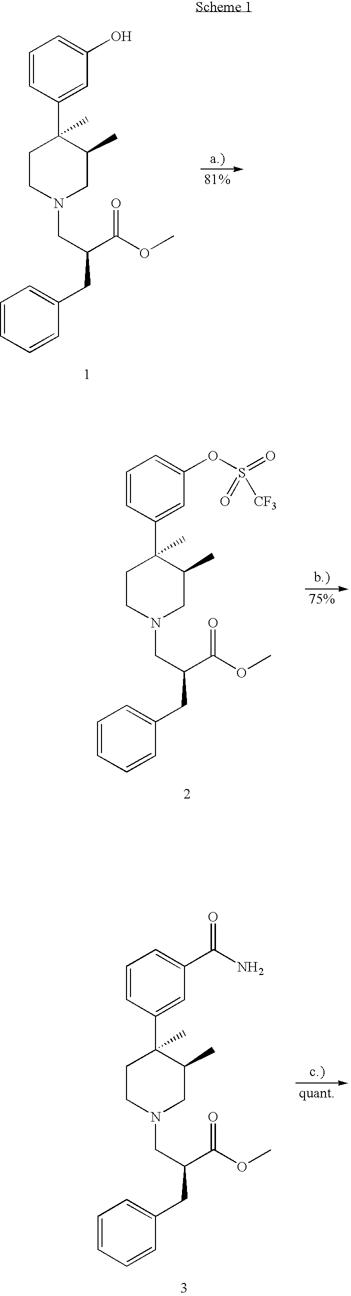

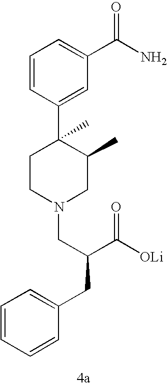

### a. Preparation of (S)-methyl 2-benzyl-3-((3R,4R)-3,4-dimethyl-4-(3-(trifluoromethyl-sulfonyloxy)phenyl)piperidin-1-yl)propanoate (2)

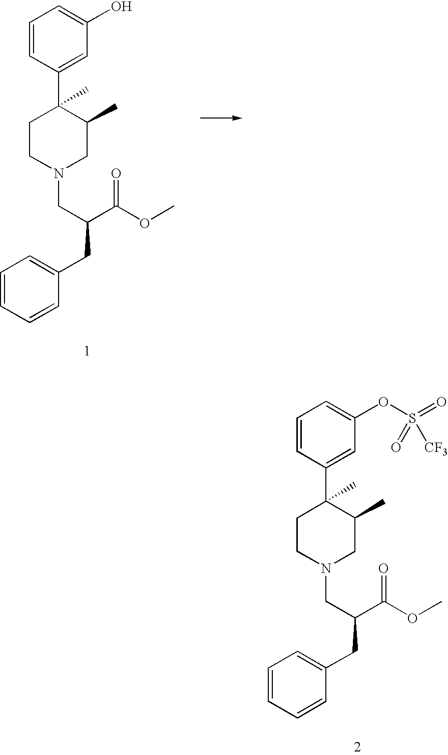

To a solution of (S)-2-benzyl-3-[(3R,4R)-4-(3-hydroxyphenyl)-3,4-dimethylpiperidin-1-yl]propionic acid methyl ester (1) (5.00 g, 0.0131 mol) (see Werner et al., J. Org. Chem., 1996, 61, 587-597) in methylene chloride (70 mL, 1 mol) was added triethylamine (3.03 mL, 0.0218 mol), followed by dropwise addition of N-phenylbis-(trifluoromethanesulfonimide) (7.02 g, 0.0196 mol) in methylene chloride (70 mL). The mixture was stirred at room temperature overnight. LCMS indicated the reaction was complete. NaOH (1N) was added and the mixture was stirred for 30 minutes. The resulting layers were separated and the aqueous layer was extracted with DCM. The organic layers were combined and washed with NaOH (1N), dried (Na₂SO₄), filtered and concentrated in vacuo. The crude reaction product was purified by silica gel chromatography and afforded the product (S)-methyl 2-benzyl-3-((3R,4R)-3,4-dimethyl-4-(3-(trifluoromethyl-sulfonyloxy)phenyl)piperidin-1-yl)propanoate (2) as a colorless oil (5.46 g, 81%). ¹H NMR (CDCl₃), δ 0.68 (d, J=7 Hz, 3H), 1.29 (s, 3H), 1.55 (m, 1H), 1.96 (m, 1H), 2.25 (m, 1H), 2.39 (m, 2H), 2.47 (m, 1H), 2.68 (m, 2H), 2.79 (m, 2H), 2.93 (m, 2H), 3.55 (s, 3H), 7.08 (dd, J=7 Hz and 2 Hz, 1H), 7.15 (m, 4H), 7.21 (m, 1H), 7.28 (m, 2H), 7.38 (t, J=8 Hz, 1H). Mass Spectral Analysis, m/z 514 [M+H]⁺

### b. Preparation of (S)-methyl 2-benzyl-3-((3R,4R)-4-(3-carbamoylphenyl)-3,4-dimethylpiperidin-1-yl)propanoate (3)

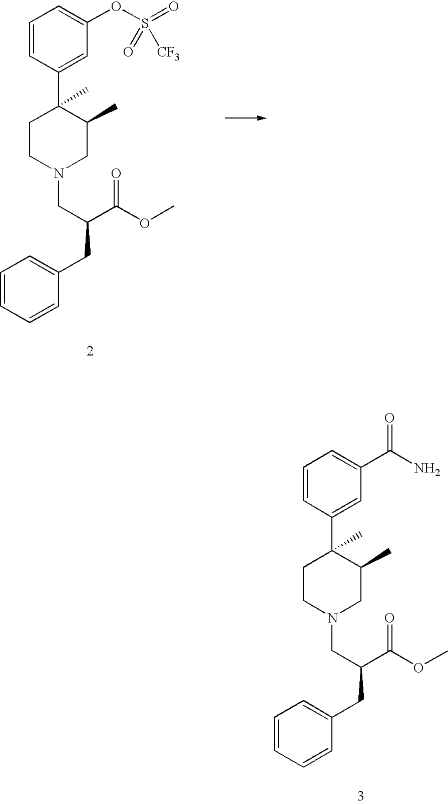

A mixture of (S)-methyl 2-benzyl-3-((3R,4R)-3,4-dimethyl-4-(3-(trifluoromethyl-sulfonyloxy)phenyl)piperidin-1-yl)propanoate (2) (5.46 g, 0.0106 mol), palladium(II) chloride (100 mg, 0.0006 mol), 1,3-bis(diphenylphosphino)propane (530 mg, 0.0013 mol) and hexamethyldisilazane (8.97 mL, 0.0425 mol) in N,N-dimethylformamide (70 mL, 0.9 mol) was purged with CO for 5 minutes and then stirred under an atmosphere of carbon monoxide at 80° C. for 1 hour. To this mixture were then added palladium acetate (200 mg, 0.001 mol) and 1,3-bis(diphenylphosphino)propane (880 mg, 0.0021 mol) and the resulting mixture was purged with CO for 10 minutes and heated at 90° C. under an atmosphere of carbon monoxide overnight. LCMS indicated a complete reaction. The reaction mixture was poured into 1N HCl solution and was extracted with ethyl acetate. The organic fractions were set aside, and the aqueous layer was made basic with NaOH (50% w/w) and extracted with ethyl acetate. The organic fractions were combined and washed with brine, dried (Na₂SO₄), filtered and evaporated. Combi flash chromatography (40 g column, 0% EtOAc in hexanes, 0→1 min; 0→50%, 1→21 min; 50%, 21→30 min) afforded (S)-methyl 2-benzyl-3-((3R,4R)-4-(3-carbamoylphenyl)-3,4-dimethylpiperidin-1-yl)propanoate (3) as a pale yellow oil (3.27 g, 75.3%). ¹H NMR (CDCl₃), δ 0.68 (d, J=7 Hz, 3H), 1.31 (s, 3H), 1.61 (m, 1H), 2.03 (m, 1H), 2.32 (m, 1H), 2.37 (m, 2H), 2.44 (m, 1H), 2.51 (dd, J=11 Hz and 3 Hz, 1H), 2.66 (m, 1H), 2.72 (m, 1H), 2.80 (m, 2H), 2.93 (m, 1H), 3.55 (s, 3H), 5.54 (br s, 1H), 6.04 (br s, 1H), 7.17 (m, 2H), 7.21 (m, 1H), 7.30 (m, 1H), 7.39 (d, J=7 Hz, 1H), 7.44 (m, 1H), 7.55 (m, 1H), 7.76 (t, J=2 Hz, 1H), 8.02 (br s, 1H). Mass Spectral Analysis, m/z 409 [M+H]⁺

### c. Preparation of (S)-2-benzyl-3-((3R,4R)-4-(3-carbamoylphenyl)-3,4-dimethylpiperidin-1-yl)propanoic acid, lithium salt (4a)

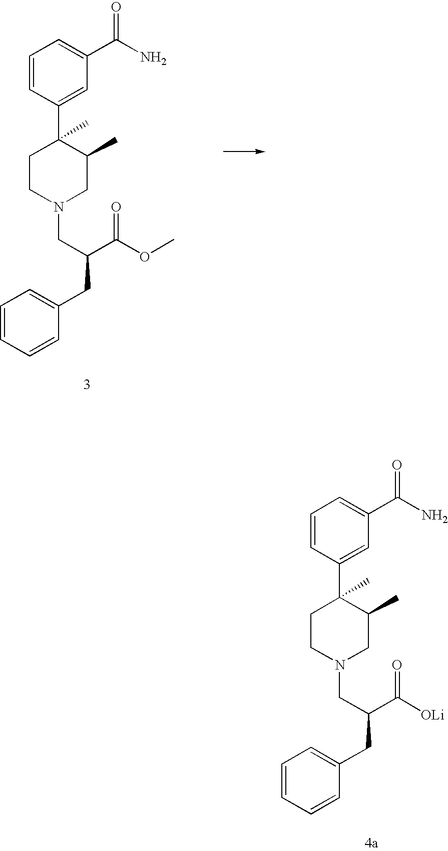

To a solution of (S)-methyl 2-benzyl-3-((3R,4R)-4-(3-carbamoylphenyl)-3,4-dimethylpiperidin-1-yl)propanoate (3) (1.50 g, 0.00367 mol) in tetrahydrofuran (20 mL, 0.2 mol) was added methanol (32 mL, 0.80 mol) and a solution of lithium hydroxide monohydrate (460 mg, 0.011 mol) in water (8 mL, 0.4 mol). The resulting mixture was stirred at room temperature overnight. LCMS indicated the reaction was complete. The solvents were evaporated, and the product lithium (S)-2-benzyl-3-((3R,4R)-4-(3-carbamoylphenyl)-3,4-dimethylpiperidin-1-yl)propanoate (4a) was obtained as a pale yellow solid (1.47 g, 100%). ¹H NMR (DMSO), δ 0.70 (d, J=7 Hz, 3H), 1.25 (s, 3H), 1.58 (d, J=10 Hz, 1H), 2.04 (m, 1H), 2.18 (t, J=11 Hz, 2H), 2.23 (dd, J=13 Hz and 11 Hz, 1H), 2.45 (m, 3H), 2.55 (m, 1H), 2.80 (m, 3H), 7.09 (m, 1H), 7.20 (m, 5H), 7.37 (t, J=8 Hz, 2H), 7.44 (d, J=8 Hz, 1H), 7.68 (d, J=7 Hz, 1H), 7.79 (br s, 1H), 8.02 (br s, 1H). Mass Spectral Analysis, m/z 395 [M−Li+2H]⁺

### Example 2

### Preparation of (S)-2-benzyl-3-((3R,4R)-4-(3-carbamoylphenyl)-3,4-dimethylpiperidin-1-yl)propanoic acid, sodium salt (4b)

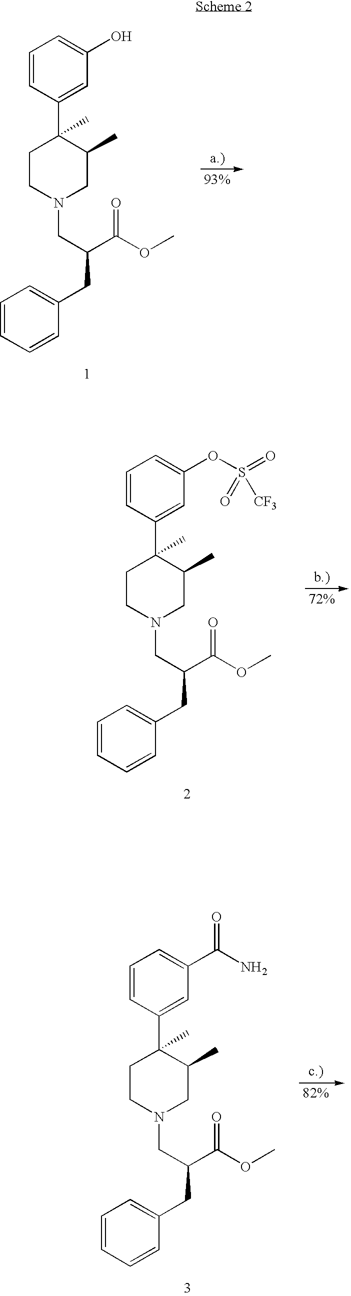

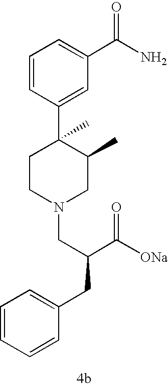

### a. Preparation of (S)-methyl-2-benzyl-3-((3R,4R)-3,4-dimethyl-4-(3-(trifluoromethyl-sulfonyloxy)phenyl)piperidin-1-yl)propanoate (2)

Example 1a was repeated, except that the reaction was scaled-up and employed 40.0 g (0.1 mol) of (S)-2-benzyl-3-[(3R,4R)-4-(3-hydroxyphenyl)-3,4-dimethylpiperidin-1-yl]propionic acid methyl ester (1) in methylene chloride (560 mL), triethylamine (24.25 mL, 0.174 mol), and N-phenylbis(trifluoromethanesulphonimide) (56.2 g, 0.157 mol) in methylene chloride (70 mL). 50 g (93%) of (S)-methyl-2-benzyl-3-((3R,4R)-3,4-dimethyl-4-(3-(trifluoromethyl-sulfonyloxy)phenyl)piperidin-1-yl)propanoate (2) was obtained as a light yellow oil. ¹H NMR (CDCl₃), δ 0.68 (d, J=7 Hz, 3H), 1.29 (s, 3H), 1.55 (m, 1H), 1.96 (m, 1H), 2.25 (m, 1H), 2.39 (m, 2H), 2.47 (m, 1H), 2.68 (m, 2H), 2.79 (m, 2H), 2.93 (m, 2H), 3.55 (s, 3H), 7.08 (dd, J=7 Hz and 2 Hz, 1H), 7.15 (m, 4H), 7.21 (m, 1H), 7.28 (m, 2H), 7.38 (t, J=8 Hz, 1H). Mass Spectral Analysis, m/z 514 [M+H]⁺

### b. Preparation of (S)-methyl 2-benzyl-3-((3R,4R)-4-(3-carbamoylphenyl)-3,4-dimethylpiperidin-1-yl)propanoate (3)

Example 1b was repeated, except that the reaction was scaled-up and employed 43.68 g (0.085 mol) of (S)-methyl 2-benzyl-3-((3R,4R)-3,4-dimethyl-4-(3-(trifluoromethyl-sulfonyloxy)phenyl)piperidin-1-yl)propanoate (2), palladium(II) chloride (0.8 g, 0.004 mol), 1,3-bis(diphenylphosphino)propane (4.24 g, 0.0103 mol) and hexamethyldisilazane (72 mL, 0.34 mol) in N,N-dimethylformamide (560 mL), palladium acetate (1.60 g, 0.007 mol) and 1,3-bis(diphenylphosphino)propane (7.04 g, 0.017 mol). 24.9 g (71.6%) of (S)-methyl 2-benzyl-3-((3R,4R)-4-(3-carbamoylphenyl)-3,4-dimethylpiperidin-1-yl)propanoate (3) was obtained as a light yellow oil. ¹H NMR (CDCl₃), δ 0.68 (d, J=7 Hz, 3H), 1.31 (s, 3H), 1.61 (m, 1H), 2.03 (m, 1H), 2.32 (m, 1H), 2.37 (m, 2H), 2.44 (m, 1H), 2.51 (dd, J=11 Hz and 3 Hz, 1H), 2.66 (m, 1H), 2.72 (m, 1H), 2.80 (m, 2H), 2.93 (m, 1H), 3.55 (s, 3H), 5.54 (br s, 1H), 6.04 (br s, 1H), 7.17 (m, 2H), 7.21 (m, 1H), 7.30 (m, 1H), 7.39 (d, J=7 Hz, 1H), 7.44 (m, 1H), 7.55 (m, 1H), 7.76 (t, J=2 Hz, 1H), 8.02 (br s, 1H). Mass Spectral Analysis, m/z 409 [M+H]⁺

### c. Preparation of (S)-2-benzyl-3-((3R,4R)-4-(3-carbamoylphenyl)-3,4-dimethylpiperidin-1-yl)propanoic acid, sodium salt (4b)

To a solution of (S)-methyl 2-benzyl-3-((3R,4R)-4-(3-carbamoylphenyl)-3,4-dimethylpiperidin-1-yl)propanoate (3) (23 g, 0.056 mol) in tetrahydrofuran (300 mL) were added methanol (300 mL) and a solution of sodium hydroxide (6.6 g, 0.16 mol) in water (100 mL). The mixture was stirred overnight at room temperature. LCMS indicated that the reaction was complete. Column chromatography with 330 g of pre-packed column and CH₃CN/CH₃OH (v/v) (1:1) as eluents afforded 20 g (82%) of the product sodium (S)-2-benzyl-3-((3R,4R)-4-(3-carbamoylphenyl)-3,4-dimethylpiperidin-1-yl)propanoate (4b) as a white solid. ¹H NMR (DMSO), δ 0.70 (d, J=7 Hz, 3H), 1.25 (s, 3H), 1.58 (d, J=10 Hz, 1H), 2.04 (m, 1H), 2.18 (t, J=11 Hz, 2H), 2.23 (dd, J=13 Hz and 11 Hz, 1H), 2.45 (m, 3H), 2.55 (m, 1H), 2.80 (m, 3H), 7.09 (m, 1H), 7.20 (m, 5H), 7.37 (t, J=8 Hz, 2H), 7.44 (d, J=8 Hz, 1H), 7.68 (d, J=7 Hz, 1H), 7.81 (br s, 1H), 8.07 (br s, 1H). Mass Spectral Analysis, m/z 395 [M−Na+2H]⁺

### Example 3

### Preparation of (S)-2-benzyl-3-((3R,4R)-4-(3-carbamoylphenyl)-3,4-dimethylpiperidin-1-yl)propanoic acid, trifluoroacetate salt (4c)

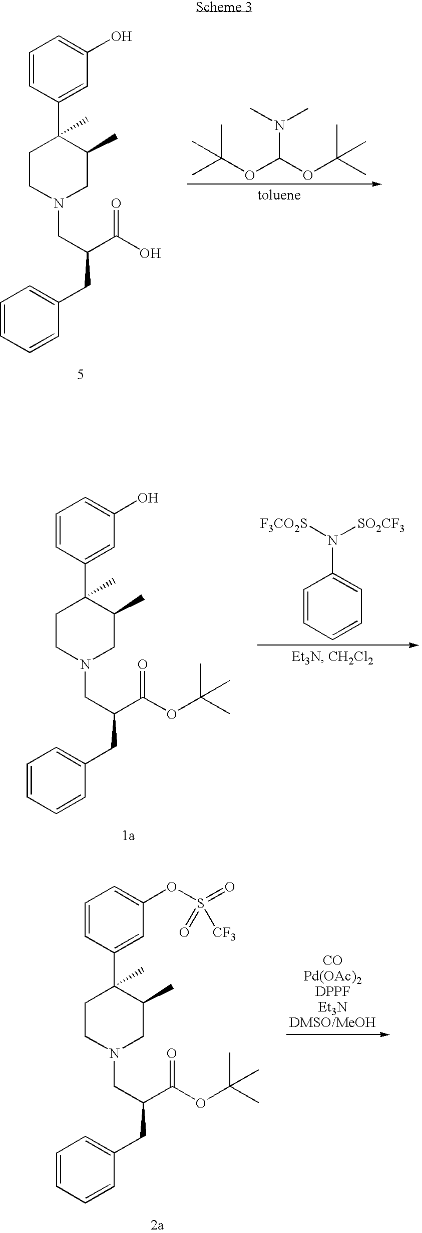

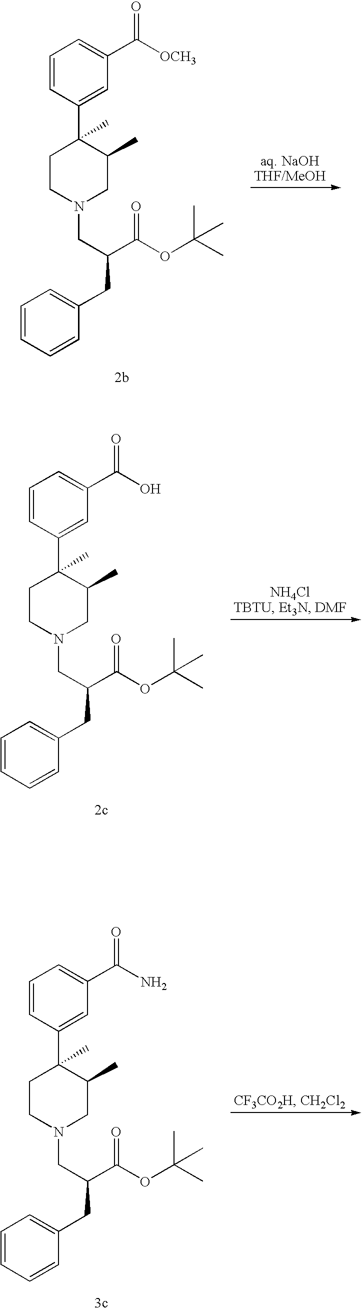

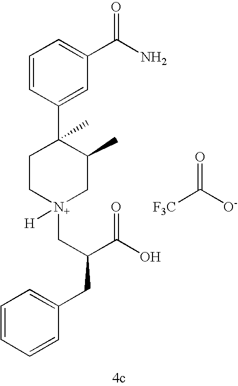

### a. Preparation of (S)-tert-butyl 2-benzyl-3-((3R,4R)-4-(3-hydroxyphenyl)-3,4-dimethylpiperidin-1-yl)propanoate (1a)

Di-tert-butoxy-N,N-dimethylmethanamine (30 mL, 0.1. mol, 4 equiv) was added dropwise over a 1 h period to a suspension of (S)-2-benzyl-3-((3R,4R)-4-(3-hydroxyphenyl)-3,4-dimethylpiperidin-1-yl)propanoic acid (5) (see Werner et al., J. Org. Chem. 1996, 61, 587-597) (10.3 g, 0.028803 mol, 1 equiv) in refluxing toluene (100 mL). The mixture was heated to reflux for an additional 8 h. The mixture was then cooled to room temperature, poured into a 1N aqueous solution of sodium hydroxide and extracted. The crude product was purified by column chromatography (hexane/ethyl acetate 8:2) to provide compound 1a (3.65 g, 31%). ¹H NMR (DMSO), δ 0.65 (d, J=7 Hz, 3H), 1.20 (s, 3H), 1.23 (s, 9H), 1.48 (d, J=13 Hz, 1H), 1.92 (dd, J=7 Hz and 4 Hz, 1H), 2.10 (m, 1H), 2.28 (m, 1H), 2.40 (m, 2H), 2.66 (m, 6H), 6.54 (dd, J=8 Hz and 1 Hz, 1H), 6.67 (m, 2H), 7.07 (t, J=8 Hz, 1H), 7.19 (m, 3H), 7.25 (m, 2H), 9.27 (br s, 1H). Mass spectral analysis: m/z=424.2 [M+H]⁺

### b. Preparation of (S)-tert-butyl 2-benzyl-3-((3R,4R)-3,4-dimethyl-4-(3-(trifluoromethylsulfonyloxy)phenyl)piperidin-1-yl)propanoate (2a)

To a cold (0° C.) suspension of compound 1a (3.65 g, 0.00862 mol, 1 equiv) and triethylamine (2.9 mL, 0.021 mol, 2.4 equiv) in anhydrous dichloromethane (60 mL) was added N-triphenyltrifluoromethane sulfonimide (3.4 g, 0.0095 mol, 1.1 equiv). The mixture was allowed to warm slowly to room temperature and stirring was continued for 12 h. The mixture was washed with an aqueous saturated sodium hydrogenocarbonate solution, and brine. The organic layer was dried over sodium sulfate and concentrated under vacuum to furnish the crude product. Purification by column chromatography (eluent: dichloromethane) afforded product 2a (3.66 g, 76%). ¹H NMR (CDCl₃), δ 0.73 (d, J=7 Hz, 3H), 1.30 (s, 12H), 1.57 (dd, J=13 Hz and 1 Hz, 1H), 1.97 (m, 1H), 2.24 (dt, J=17 Hz and 6 Hz, 1H), 2.33 (dd, J=12 Hz and 5 Hz, 1H), 2.43 (dt, J=15 Hz and 3 Hz, 1H), 2.51 (dd, J=11 Hz and 3 Hz, 1H), 2.65 (m, 1H), 2.70 (m, 1H), 2.79 (m, 4H), 7.08 (dd, J=8 Hz and 3 Hz, 1H), 7.17 (m, 4H), 7.28 (m, 3H), 7.38 (t, J=8 Hz, 1H). Mass spectral analysis: m/z=556.2 [M+H]⁺

### c. Preparation of methyl 3-((3R,4R)-1-(S)-2-benzyl-3-tert-butoxy-3-oxopropyl)-3,4-dimethylpiperidin-4-yl)benzoate (2b)

To a stirred solution of compound 2a (3.66 g, 0.00659 mol, 1 equiv) in a mixture of methanol (20 mL) and dimethylsulfoxide (25 mL) was added triethylamine (2.0 mL, 0.014 mol, 2.2 equiv). Carbon monoxide gas was bubbled through the mixture for 5 minutes. To the mixture was added palladium (II) acetate (0.1 g, 0.0006 mol, 0.1 eq) followed by 1,1′-bis(diphenylphosphino)ferrocene (0.7 g, 0.001 mol, 0.2 equiv). Carbon monoxide gas was bubbled through the mixture for 15 minutes and it was then stirred under an atmosphere of carbon monoxide and heated at 65° C. overnight. The mixture was cooled to room temperature and poured into water. The mixture was extracted with diethyl ether and the combined organic extracts were dried over sodium sulfate. Evaporation of the solvent under vacuum afforded an oil which was purified by column chromatography (eluent: hexane/ethyl acetate mixtures of increasing polarity) to afford compound 2b (2.23 g, 73%). Mass spectral analysis: m/z 466.2 [M+H]⁺

### d. Preparation of 3-((3R,4R)-1-((S)-2-benzyl-3-tert-butoxy-3-oxopropyl)-3,4-dimethylpiperidin-4-yl)benzoic acid (2c)

An aqueous 6N solution of sodium hydroxide (1 mL, 6 equiv) was added to a solution of compound 2b (0.430 g, 0.000923 mol, 1 equiv) in tetrahydrofuran (10 mL) and methanol (2 mL). The mixture was stirred at room temperature for 12 h and then neutralized to pH ˜7 using a 6N solution of hydrochloric acid. The mixture was concentrated under reduced pressure. A solution of dichloromethane/methanol (95:5) was added to the mixture and the resulting suspension was filtered. The filtrate was concentrated under reduced pressure, and the crude product 2c (0.340 g, 81%) was used in the next step without further purification. ¹H NMR (CDCl₃), δ 0.73 (d, J=7 Hz, 3H), 1.29 (s, 9H), 1.30 (s, 3H), 1.64 (q, J=12 Hz, 1H), 2.05 (m, 1H), 2.32 (m, 1H), 2.39 (m, 1H), 2.45 (m, 1H), 2.55 (dd, J=11 Hz and 2 Hz, 1H), 2.68 (m, 1H), 2.73 (t, J=11 Hz and 10 Hz, 1H), 2.80 (m, 4H), 7.19 (m, 3H), 7.25 (m, 2H), 7.37 (t, J=8 Hz, 1H), 7.47 (d, J=7 Hz, 1H), 7.90 (d, J=8 Hz, 1H), 8.00 (s, 1H). Mass spectral analysis: m/z 450.3 [M−H]⁺

### e. Preparation of (S)-tert-butyl 2-benzyl-3-((3R,4R)-4-(3-carbamoylphenyl)-3,4-dimethylpiperidin-1-yl)propanoate (3c)

To a suspension of product 2c (0.340 g, 0.000753 mol, 1 equiv), triethylamine (0.6 mL, 0.004 mol, 6 equiv), and ammonium chloride (0.2 g, 0.004 mol, 5 equiv) in dimethylformamide (5 mL) was added TBTU (0.36 g, 0.0011 mol, 1.5 equiv). This mixture was stirred for 12 h at room temperature under a nitrogen atmosphere, poured into brine and extracted with ethyl acetate. The organic layer was separated, washed with water, dried (sodium sulfate), filtered and concentrated. The crude product was purified by column chromatography (eluent: hexane/ethyl acetate mixtures of increasing polarity) to afford compound 3c (0.230 g, 67%). ¹H NMR (CDCl₃), δ 0.73 (d, J=7 Hz, 3H), 1.30 (s, 9H), 1.31 (s, 3H), 1.63 (dd, J=12 Hz and 1 Hz, 1H), 2.04 (m, 1H), 2.32 (m, 2H), 2.43 (m, 1H), 2.52 (dd, J=11 Hz and 3 Hz, 1H), 2.66 (d, J=11 Hz, 1H), 2.70 (m, 1H), 2.80 (d, 4H), 7.19 (m, 3H), 7.25 (m, 2H), 7.38 (t, J=8 Hz, 1H), 7.45 (m, 1H), 7.55 (m, 1H), 7.77 (s, 1H). Mass spectral analysis: m/z 451.2 [M+H]⁺

### f. Preparation of (S)-2-benzyl-3-((3R,4R)-4-(3-carbamoylphenyl)-3,4-dimethylpiperidin-1-yl)propanoic acid, trifluoroacetic acid salt (4c)

Trifluoroacetic acid (1.5 mL, 0.019 mol, 38 equiv) was added dropwise to a solution of compound 3c (0.230 g, 0.000510 mol, 1 equiv) in dichloromethane (10 mL). The reaction mixture was stirred at room temperature for 12 h. The mixture was concentrated under reduced pressure and the crude product was purified by HPLC to provide TFA salt 4c (0.053 g, 20%). ¹H NMR (DMSO), δ 0.66 (br s, 3H), 1.37 (s, 3H), 1.93 (d, J=15 Hz, 1H), 2.38 (m, 2H), 2.90 (d, J=7 Hz, 2H), 3.28 (m, 3H), 3.44 (m, 4H), 7.26 (d, J=7 Hz, 2H), 7.33 (m, 2H), 7.43 (m, 2H), 7.73 (d, J=7 Hz, 1H), 7.79 (s, 1H), 8.02 (s, 1H), 8.76 (br s, 0.5H), 13.14 (br s, 0.5H). Mass spectral analysis: m/z 395.2 [M+H]⁺

### Example 4

### Preparation of (S)-2-Benzyl-3-((3R,4R)-4-(3-carbamoylphenyl)-3,4-dimethylpiperidin-1-yl)propanoic acid (4d)

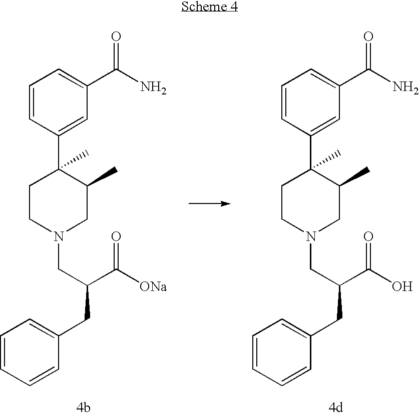

Cation exchange resin (AG 50W-X8 resin from BioRad, 4 g) was stirred in of distilled water (20 mL) for 10 min and filled into a glass column. Aqueous HCl (50 mL, 1N) was passed through the column, then the column was washed with water until the eluent was close to neutral pH. Sodium (S)-2-benzyl-3-((3R,4R)-4-(3-carbamoylphenyl)-3,4-dimethylpiperidin-1-yl)propanoate (4b) (200 mg) was dissolved in water (15 mL) and the resulting solution was passed through the column. The column was washed with distilled water and the eluents were combined. Lyophillization of the aqueous eluents provided (S)-2-benzyl-3-((3R,4R)-4-(3-carbamoylphenyl)-3,4-dimethylpiperidin-1-yl)propanoic acid (4d) as a white powder. ¹H NMR (DMSO), d 0.62 (d, J=7 Hz, 3H), 1.27 (s, 3H), 1.63 (d, J=13 Hz, 1H), 2.10 (d, J=6 Hz, 1H), 2.24 (m, 1H), 2.40 (m, 2H), 2.57 (m, 2H), 2.68 (m, 3H), 2.79 (m, 1H), 2.92 (m, 1H), 7.21 (m, 3H), 7.29 (m, 2H), 7.37 (m, 2H), 7.44 (d, J=8 Hz, 1H), 7.68 (d, J=8 Hz, 1H), 7.78 (s, 1H), 7.99 (s, 1H). Mass Spectral Analysis, m/z 395 [M+H]⁺

### Example 5

### Preparation of (S)-2-benzyl-3-((3R,4R)-4-(3-carbamoylphenyl)-3,4-dimethylpiperidin-1-yl)propanoic acid, oxalic acid salt (4e)

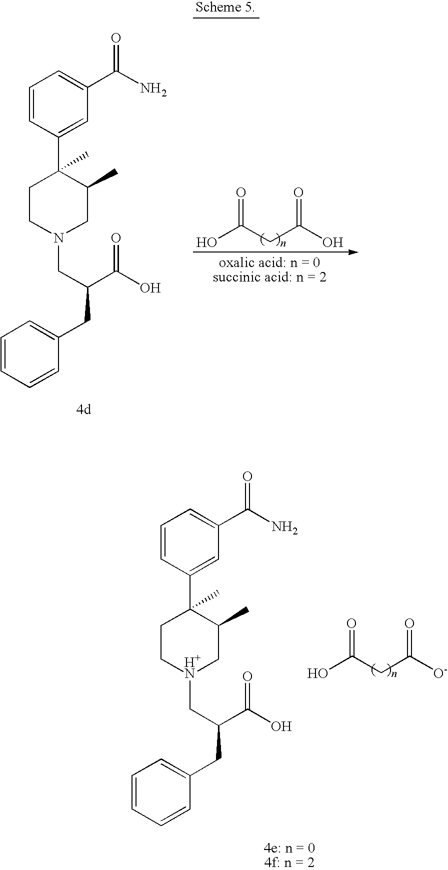

Compound 4d was dissolved in methanol. To this was added 1 equivalent of oxalic acid dissolved in isopropyl alcohol (Scheme 5). The mixture was stirred at room temperature under nitrogen overnight. The solvent was evaporated, a 5:1 water/acetone mixture was added, stirred, the acetone was removed and the remaining aqueous solution was lyophilized to give 4e. ¹H NMR (DMSO), δ 0.65 (d, J=7 Hz, 3H), 1.30 (s, 3H), 1.72 (m, 1H), 2.25 (m, 2H), 2.45 (m, 1H), 2.55 (m, 1H), 2.67 (m, 1H), 2.75 (m, 2H), 2.86 (m, 2H), 2.98 (m, 2H), 7.21 (m, 3H), 7.29 (t, J=7 Hz, 2H), 7.39 (m, 3H), 7.70 (d, J=8 Hz, 1H), 7.78 (s, 1H), 7.99 (br s, 1H). Elemental analysis (CHN): C₂₆H₃₂N₂O₇ 1.5H₂O. Theory: C, 61.04; H, 6.90. N, 5.48. Found: C, 60.82; H, 6.70; N, 5.34.

### Example 6

### Preparation of (S)-2-benzyl-3-((3R,4R)-4-(3-carbamoylphenyl)-3,4-dimethylpiperidin-1-yl)propanoic acid, succinic acid salt (4f)

Compound 4d was dissolved in methanol. To this was added 1 equivalent of succinic acid dissolved in isopropyl alcohol (Scheme 5). The mixture was stirred at room temperature under nitrogen overnight. The solvent was evaporated, a 5:1 water/acetone mixture was added, stirred, the acetone was removed and the remaining aqueous solution was lyophilized to give 4f. ¹H NMR (DMSO), δ 0.62 (d, J=7 Hz, 3H), 1.27 (s, 3H), 1.63 (m, 1H), 2.12 (m, 2H), 2.25 (m, 1H), 2.45 (m, 1H), 2.66 (m, 1H), 2.70 (m, 2H), 2.78 (m, 2H), 2.80 (m, 2H), 7.17 (m, 3H), 7.26 (t, J=7 Hz, 2H), 7.43 (m, 3H), 7.67 (d, J=8 Hz, 1H), 7.77 (s, 1H), 7.96 (br s, 1H). Elemental analysis (CHN): C₂₈H₃₆N₂O₇ 1.0H₂O. Theory: C, 63.38; H, 7.22. N, 5.28. Found: C, 63.38; H, 7.07; N, 5.22.

The following comparative examples and biological testing data illustrate the surprisingly and unexpectedly improved biological and pharmacokinetic profiles of compounds and salts thereof according to the present invention in comparison to compounds of the prior art. In particular, the following comparative examples and comparative biological testing data demonstrate the desirable affinities of compounds of the invention for opioid receptors, as well as their advantageous ability to antagonize μ opioid receptors. Compounds of the present invention also demonstrate desirably favorable predictability and reduced variability in in vivo pharmacokinetic behavior, as well as advantageously improved bioavailability. In view of this improved profile of biological activities, the present compounds and salts thereof are particularly useful in the treatment of diseases or disorders that are associated with opioid receptors including, for example, gastrointestinal dysfunction and side effects associated with opioids. The improved profile of biological activities of compounds and salts of the present invention as compared to prior art compounds is surprising and unexpected.

### COMPARATIVE EXAMPLES

Comparative Examples C-1, C-2, and C-3 describe the preparation of prior art compounds. These compounds were prepared according to procedures outlined in Schemes 6 and 7.

Palladium catalyzed carbonylation of 2 afforded the diester 10 which was converted to the dicarboxylic acid 11 (Example C-1) by basic cleavage of the two methyl ester protecting groups in diester 10 (see Scheme 6). Palladium catalyzed carbamoylation of 13 afforded amide ester 14. Treatment of intermediate 14 with acid yielded 15 (Example C-2). Treatment of intermediate 14 with acid, followed by adjustment of the pH of the reaction mixture to pH 8 with aqueous sodium hydroxide provided sodium salt 16 (Example C-3) (see Scheme 7).

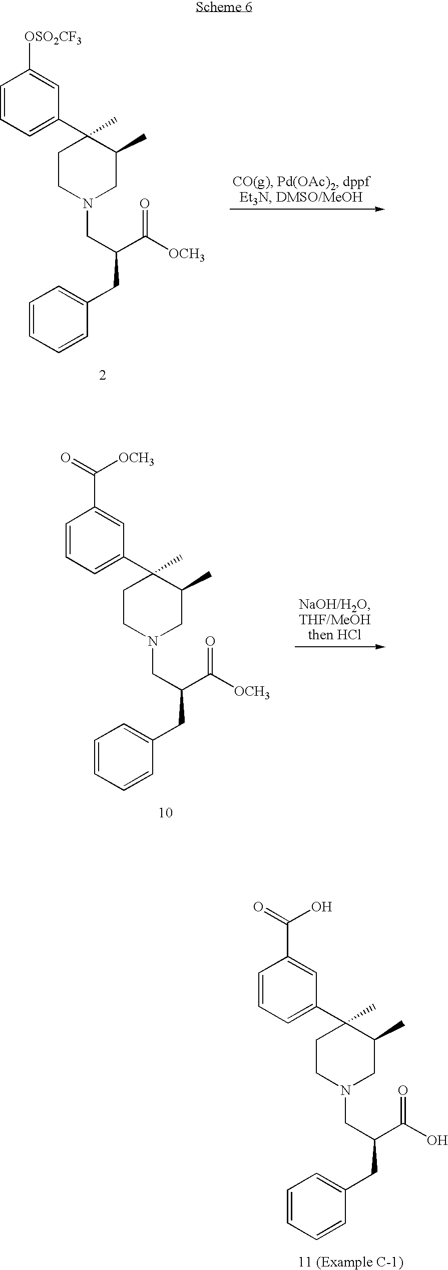

### Example C-1

### 3-[1-(2S-Carboxy-3-phenyl-propyl)-3R,4R-dimethyl-piperidin-4-yl]-benzoic acid (11)

To a stirred solution of compound 2 (1.72 g, 0.0033 mol, 1 eq) in a mixture of methanol (15 mL) and dimethylsulfoxide (20 mL) was added triethylamine (1.03 mL, 0.0073 mol, 2.2 eq). Carbon monoxide gas was bubbled through the mixture for 5 minutes. To the mixture was added palladium (II) acetate (0.075 g, 0.00033 mol, 0.1 eq) followed by 1,1′-bis(diphenylphosphino)ferrocene (0.371 g, 0.00067 mol, 0.2 eq). Carbon monoxide gas was bubbled through the mixture for 15 minutes and the mixture was then stirred under an atmosphere of carbon monoxide and heated at 65° C. overnight. The mixture was cooled to room temperature and poured into water (100 mL). The mixture was extracted with ethyl acetate (3×50 mL). The organic extracts were combined and washed with water (100 mL), brine (100 mL) and dried over sodium sulfate. Evaporation of the solvent under vacuum afforded an oil. The crude oil product was purified by column chromatography (eluent: hexane/ethyl acetate 95:5) to afford compound 10 (0.720 g, 51%); mass spectral analysis: m/z 424 [M+H]⁺.

A solution of aqueous 2 N sodium hydroxide (2.55 mL, 0.00509 mol, 6 eq) was added dropwise to a cold (0° C.) solution of 10 (0.360 g, 0.00084 mol, 1 eq) in tetrahydrofuran (10 mL). The mixture was allowed to warm to room temperature and stirring was continued for 5 hours. A solution of lithium hydroxide monohydrate (0.213 g, 0.0050 mol, 6 eq) in water (5 mL) was added to the mixture (methanol (3 mL) was added for solubilization) and stirring was continued for 12 hours. A 12N aqueous HCl solution (0.8 mL) was added to neutralize the mixture which was concentrated under vacuum. The precipitate was collected by filtration and washed with diethyl ether to provide 3-[1-(2S-carboxy-3-phenyl-propyl)-3R,4R-dimethyl-piperidin-4-yl]-benzoic acid (11) as a white solid (0.2 g, 64%); mass spectral analysis: m/z 396 [M+H]⁺

The preparation of carboxamides 13 and 14 are outlined in Scheme 7. Glycine tert-butyl ester 12, prepared from acid 5 according to the procedure described by Werner et al. (J. Org. Chem., 1996, 61, 587-597) was converted to the triflate 13 using N-phenyltrifluoro-methane sulfonamide using the reaction conditions described above for the preparation of triflate 2. Palladium catalyzed formation of the carboxamide 14 from the triflate 13 was conducted in the presence of carbon monoxide and (TMS)₂NH. Acidic hydrolysis of 14 afforded compound 15 (Example C-2) which was isolated as its TFA salt after purification by preparative HPLC. Alternatively, after acidic hydrolysis of compound 14, the pH of the solution was adjusted to pH 8 and lyophilized to afford compound 16 (Example C-3).

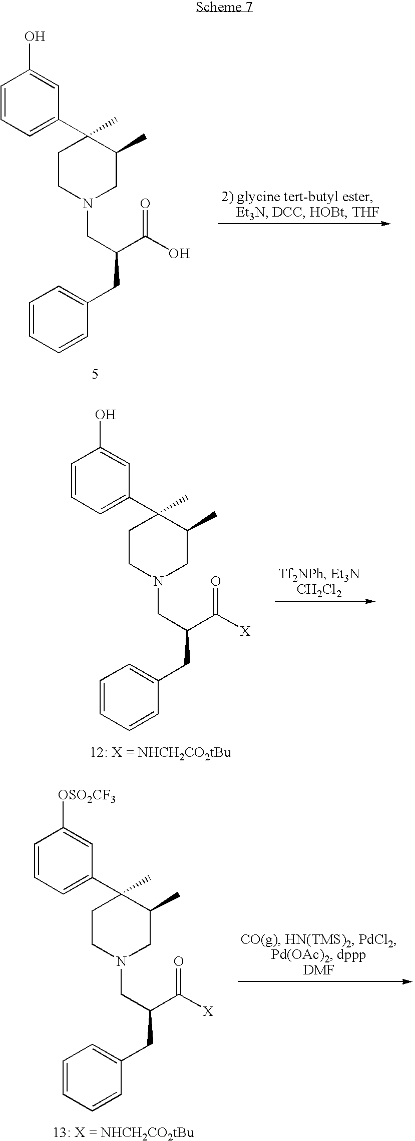

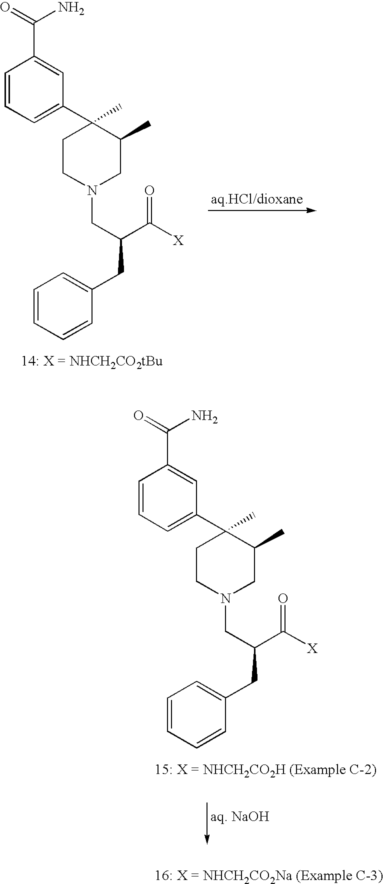

### Example C-2

### Preparation of [[2(R)-[[4(R)-(3-Amidophenyl)-3(R),4-dimethyl-1-piperidinyl]methyl]-1-oxo-3-phenylpropyl]amino]acetic acid, trifluoromethylacetate salt (15)

A stirred solution of compound 13 (0.5 g, 0.816 mmol, 1 eq), palladium chloride (9 mg, 6 mol %, 48.96 μmol), diphenylphosphinopropane (39 mg, 12 mol %, 97.9 μmol) and HN(TMS)₂ (0.69 mL, 3.264 mmol, 4 eq) was purged with CO(g) for 5 minutes, then stirred under an atmosphere of CO(g) for 1 hour at 80° C. After this time were added palladium acetate (18 mg, 10 mol %, 0.0816 mmol) and diphenylphosphinopropane (65 mg, 0.163 mmol). This mixture was purged with CO(g) for 10 minutes, then stirred under an atmosphere of CO(g) for 4 hours at 85-90° C. The reaction mixture was concentrated under vacuum and partitioned between dichloromethane (50 mL) and water (50 mL). The aqueous layer was extracted with dichloromethane (2×50 mL). The organic extracts were combined and washed with brine (50 mL), dried over sodium sulfate and concentrated. The crude product was purified by column chromatography (eluent: dichloromethane/methanol 97.5:2.5) to afford compound 14 as a yellow foamy solid. (0.170 g, 41%); mass spectral analysis: m/z=508 [M+H]⁺

A solution of compound 14 (0.170 g, 0.335 mmol, 1 eq) in 4N HCl in dioxane (7.5 mL) was stirred at room temperature for 2.5 hours. The solvent was removed under vacuum affording a yellow crystalline solid. The crude product was purified by preparative HPLC (methanol/water/TFA) to provide [[2(R)-[[4(R)-(3-amidophenyl)-3(R),4-dimethyl-1-piperidinyl]methyl]-1-oxo-3-phenylpropyl]amino]acetic acid (15) as the TFA salt (0.098 g, 55%); mass spectral analysis: m/z 452 [M+H]⁺

### Example C-3

### Preparation of [[2(R)-[[4(R)-(3-Amidophenyl)-3(R),4-dimethyl-1-piperidinyl]methyl]-1-oxo-3-phenylpropyl]amino]acetic acid, sodium salt (16)

A solution of {(S)-2-benzyl-3-[(3R,4R)-4-(3-carbamoylphenyl)-3,4-dimethylpiperidin-1-yl]propionyl-amino}acetic acid tert-butyl ester (14) (2.08 g, 0.00410 mol) in 6M of hydrogen chloride in water (100 mL, 0.7 mol) was stirred at room temperature overnight. LCMS indicated that the reaction was complete. The pH of the reaction mixture was adjusted to approximately neutral (˜pH 8) with 1M NaOH. The solvents were evaporated and the residue chromatographed on silica gel (MeOH/ethyl acetate 0-30%). The resulting white solid obtained was taken up in 10% methanol in DCM and the precipitate was removed by filtration. The solvents were evaporated to give the sodium salt 16 (850 mg, 46%). ¹H NMR (DMSO), d 0.63 (d, J=7 Hz, 3H), 1.24 (s, 3H), 1.55 (d, J=10 Hz, 1H), 2.03 (d, 1H), 2.21 (m, 1H), 2.30 (t, J=6 Hz, 2H), 2.41 (dd, J=11 Hz and 2 Hz, 1H), 2.53 (m, 1H), 2.63 (m, 2H), 2.79 (m, 1H), 2.85 (m, 2H), 3.52 (m, 2H), 7.16 (t, J=7 Hz, 1H), 7.23 (m, 4H), 7.35 (m, 2H), 7.43 (d, J=8 Hz, 1H), 7.67 (d, J=8 Hz, 1H), 7.78 (s, 1H), 8.00 (br s, 2H); mass spectral analysis: m/z 452.2 [M+H]⁺

### Biological Assays

The potencies of the compounds were determined by testing the ability of a range of concentrations of each compound to inhibit the binding of the non-selective opioid antagonist, [³H]diprenorphine, to the cloned human μ, κ, and δ opioid receptors, expressed in separate cell lines. IC₅₀ values were obtained by nonlinear analysis of the data using GraphPad Prism version 3.00 for Windows (GraphPad Software, San Diego). Kᵢ values were obtained by Cheng-Prusoff corrections of IC₅₀ values.

### Receptor Binding (In Vitro Assay)

The receptor binding method (DeHaven and DeHaven-Hudkins, 1998) was a modification of the method of Raynor et al. (1994). After dilution in buffer A and homogenization, membrane proteins (10-80 μg) in 250 μL were added to mixtures containing test compound and [³H]diprenorphine (0.5 to 1.0 nM, 40,000 to 50,000 dpm) in 250 μL of buffer A in 96-well deep-well polystyrene titer plates (Beckman). After incubation at room temperature for one hour, the samples were filtered through GF/B filters that had been presoaked in a solution of 0.5% (w/v) polyethylenimine and 0.1% (w/v) bovine serum albumin in water. The filters were rinsed 4 times with 1 mL of cold 50 mM Tris HCl, pH 7.8 and radioactivity remaining on the filters determined by scintillation spectroscopy. Nonspecific binding was determined by the minimum values of the titration curves and was confirmed by separate assay wells containing 10 μM naloxone. Kᵢ values were determined by Cheng-Prusoff corrections of IC₅₀ values derived from nonlinear regression fits of 12 point titration curves using GraphPad Prism® version 3.00 for Windows (GraphPad Software, San Diego, Calif.).

To determine the equilibrium dissociation constant for the inhibitors (Kᵢ), radioligand bound (cpm) in the presence of various concentrations of test compounds was measured. The concentration to give half-maximal inhibition (EC₅₀) of radioligand binding was determined from a best nonlinear regression fit to the following equation,

$$Y = Bottom + \frac{( Top - Bottom )}{1 + {10}^{X - Log E C 50}}$$

where Y is the amount of radioligand bound at each concentration of test compound, Bottom is the calculated amount of radioligand bound in the presence of an infinite concentration of test compound, Top is the calculated amount of radioligand bound in the absence of test compound, X is the logarithm of the concentration of test compound, and LogEC₅₀ is the log of the concentration of test compound where the amount of radioligand bound is half-way between Top and Bottom. The nonlinear regression fit was performed using the program Prism® (GraphPad Software, San Diego, Calif.). The K, values were then determined from the EC₅₀ values by the following equation,

$${K}_{i} = \frac{E {C}_{50}}{1 + \frac{[ ligand ]}{{K}_{d}}}$$

where [ligand] is the concentration of radioligand and K_{d} is the equilibrium dissociation constant for the radioligand.

The potencies of the antagonists were assessed by their abilities to inhibit agonist-stimulated [³⁵S]GTPγS binding to membranes containing the cloned human μ, κ, or δ opioid receptors. The agonist used for the μ opioid receptor was loperamide.

To determine the IC₅₀ value, which was the concentration to give half-maximal inhibition of agonist-stimulated [³⁵S]GTPγS binding, the amount of [³⁵S]GTPγS bound in the presence of a fixed concentration of agonist and various concentrations of antagonist was measured. The fixed concentration of agonist was the EC₈₀ for the agonist, which was the concentration to give 80% of the relative maximum stimulation of [³⁵S]GTPγS binding. The IC₅₀ value was determined from a best nonlinear regression fit of the data to the following equation,

$$Y = Bottom + \frac{( Top - Bottom )}{1 + {10}^{X - Log I C 50}}$$

where Y is the amount of [³⁵S]GTPγS bound at each concentration of antagonist, Bottom is the calculated amount of [³⁵S]GTPγS bound in the presence of an infinite concentration of antagonist, Top is the calculated amount of [³⁵S]GTPγS bound in the absence of added antagonist, X is the logarithm of the concentration of antagonist, and LogIC₅₀ is the logarithm of the concentration of antagonist where the amount of [³⁵S]GTPγS bound is halfway between Bottom and Top. The nonlinear regression fit was performed using GraphPad Prism® version 3.00 for Windows (GraphPad Software, San Diego, Calif.).

The compounds and salts thereof prepared in Examples 1 to 6, C-1, C-2 and C-3 were tested for their affinities as antagonists towards μ, κ and δ opioid receptors. The results of these binding affinity tests are summarized in Table I below.

TABLE I

| | MOR Kᵢ | DOR Kᵢ | KOR Kᵢ | DOR/MOR |
|---|---|---|---|---|
| Example | (nM) | (nM) | (nM) | Ratio |
| 1 (Li salt) | 11.2 | 340 | >10000 | 30 |
| 95% conf. | 8.4-14.8 | 278-409 | | |
| 2 (Na salt) | 17.3 | 308 | >10000 | 18 |
| 95% conf. | 13.9-21.6 | 229-415 | | |
| 3 (TFA salt) | 12.4 | 397 | >10000 | 32 |
| 95% conf. | 7.9-18.8 | 250-597 | | |
| 4 | 14 | 460 | >10000 | 33 |
| 95% conf. | 6.9-30 | 220-950 | | |
| 5 (oxalate) | 16.9 | 310 | >10000 | 18 |
| 95% conf. | 13.3-21.7 | 227-424 | | |
| 6 (succinate) | 18.1 | 527 | >10000 | 29 |
| 95% conf. | 5.6-58 | n.d. | | |
| C-1 | 2285 | >10000 | >10000 | N/A |
| 95% conf. | nd | | | |
| C-2 (TFA salt) | 2.6 | 85 | 660 | 33 |
| 95% conf. | 1.6-4.2 | 55-130 | 83-5200 | |
| C-3 (Na salt) | 2.9 | 98 | 930 | 34 |
| 95% conf. | 2.0-4.1 | 69-140 | 470-1900 | |

“MOR” refers to mu opioid receptor, “DOR” refers to delta opioid receptor and “KOR” refers to kappa opioid receptor

Antagonism of μ opioid receptors is a measure of the ability of an agent to treat gastrointestinal dysfunction that may be manifested by slowing of gastrointestinal transit due to opioid administration, as in the case, for example, of opioid-induced bowel dysfunction or opioid-induced constipation, as well as gastrointestinal disorders resulting from injury or surgery as in the case, for example, of ileus, such as post-surgical ileus or post-partum ileus.

Results of in vitro binding studies as tabulated in Table I demonstrate that compounds of the invention, as represented by Examples 1 to 6, as well as prior art compounds Examples C-2 and C-3, were potent antagonists based on their abilities to inhibit agonist-stimulated [³⁵S]GTPγS binding in vitro with IC₅₀s<100 nM at the μ receptor. With regard to relative binding strength, the in vitro binding studies indicated that prior art compounds Examples C-2 and C-3 were about 4 to about 7 times more potent than Examples 1 to 4 as antagonists of μ opioid receptors.

Prior art compound Example C-1 demonstrated significantly reduced inhibition of agonist-stimulated [35S]GTPγS binding relative to the other test compounds in the in vitro studies. Due to this significantly reduced μ opioid receptor binding affinity, Example C-1 was not evaluated for in vitro functional activity or in vivo efficacy.

### Mouse Gastrointestinal Transit (GIT) Assay (In Vivo Assay)

Male Swiss-Webster mice (25-30 g) obtained from Ace Animals (Boyertown, Pa.) were used for all experiments. Mice were housed 4/cage in polycarbonate cages with food and water available ad libitum. Mice were on a 12 hours light:dark schedule with lights on at 6:30 a.m. All experiments were performed during the light cycle. Mice were fasted the night before the experiment, with water available ad libitum.

Mice were administered vehicle (10% DMSO:20% Cremophor EL:70% saline) or test agent (3 mg/kg orally 2 or 6 hours before determination of GIT. Compounds were administered in a volume of 0.1 ml/10 g of body weight. Morphine (3 mg/kg) or vehicle (0.9% saline) was administered s.c. 35 min to induce slowed GIT, prior to determination of GIT. Ten minutes after the morphine treatment, mice were administered 0.2 ml of a charcoal meal orally. The charcoal meal consisted of a slurry of charcoal, flour, and water in the following ratio (1:2:8, w:w:v). Twenty-five minutes after receiving the charcoal meal, the mice were euthanized with CO₂ and GIT was determined.

GIT is expressed as % GIT by the following formula:

$$\frac{( distance to leading edge of charcoal meal ( cm ) )}{( total length of the small intestine ( cm ) )} \times 100.$$

For each test agent, a value for % Antagonism (% A) was determined for the 2 and 6 hr antagonist pretreatment. Using the mean % GIT for each treatment group, % A was calculated using the following formula:

$$1 - ( \frac{( \begin{matrix}mean vehicle response - \\ mean antagonist + morphine response\end{matrix} )}{( mean vehicle response - mean morphine response )} ) \times 100$$

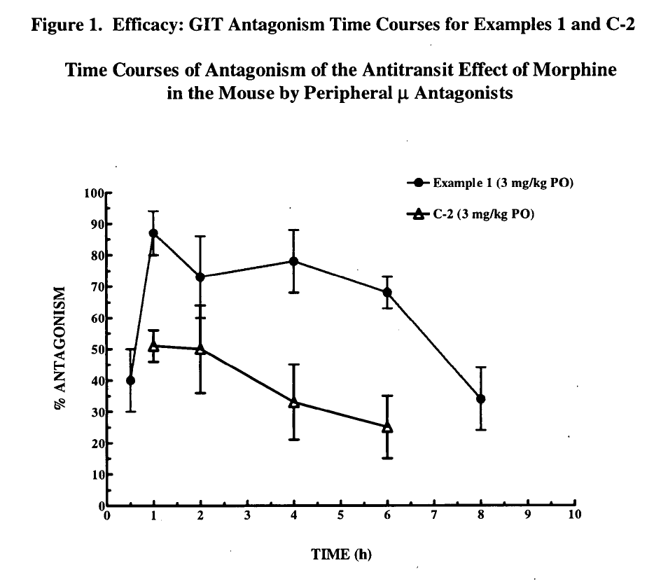

The antagonist activities of Example 1 and prior art compound Example C-2 were evaluated using the GIT (in vivo) assay. The results of these studies are depicted graphically in FIG. 1. Specifically, analysis conducted one hour after administering a 3 mg/kg dose of Example 1 demonstrated a recovery of almost 90% of the pre-opioid treatment efficacy of GIT. Analysis conducted one hour after administering a 3 mg/kg dose of Example C-2 demonstrated a recovery of 50% of the pre-opioid treatment efficacy of GIT. The improvement in GIT efficiency demonstrated with Example 1 as compared to Example C-2 was observed through the course of the 8-hour experiment until the administered agents were sufficiently metabolized to minimize their effects on opioid receptors. The area under the curve values (AUC values) were calculated for Example 1 and Example C-2, as set forth below in Table II.

TABLE II

| Example | AUC values (h * percent antagonism (0-6 h)) |
|---|---|
| 1 | 408.8 |
| C-2 | 191.5 |

As shown in Table II, the AUC value observed for Example 1 was 2.1 times greater than the AUC value for Example C-2. This doubling of the efficacy of Example 1 as compared to

Example C-2 was surprising and unexpected since C-2 was 4.3 times more potent than Example 1 as an MOR antagonist in the opioid receptor binding assay (see Table I, supra).

### Pharmacokinetic Data Procedures

### A. Rat PK Protocol

Jugular vein cannulated (JVC) male Sprague-Dawley (SD) rats (Charles River, Raleigh, N.C.) were housed individually in polycarbonate cages on alpha-dri bedding in an environmentally controlled room with a 12 hour light-dark cycle. Rats were allowed to acclimate for 3-5 days prior to the study. Animals were fasted overnight prior to drug administration and were fed after the 4 h blood collection.

### Dosing Solution Preparation

The suspension used for PO administration was prepared at a nominal concentration of 2 mg/mL of test agent in 0.5% methylcellulose with 0.1% Tween®80.

### Drug Administration and Sample Collection

Two groups of three JVC rats each received a single PO dose of 10 mg/kg. The PO dose was administered by oral gavage in a dosing volume of 5 mL/kg. Blood samples (0.6 mL) were collected via jugular vein cannula at predose, 15 and 30 minutes and 1, 2, 4, 6, 8 and 24 h postdose in EDTA-containing tubes. Plasma was obtained by centrifugation at 3000 rpm for 15 minutes. The samples were stored at −20° C. until analysis.

### Plasma Sample Preparation

Plasma aliquots were deproteinized with internal standard solution in acetonitrile. The samples were vortexed, centrifuged and an aliquot of the supernatant was mixed with water. The diluted aliquot was then analyzed.

### Sample Analysis

Plasma concentrations were determined by high performance liquid chromatography with tandem mass spectrometric detection (LC/MS/MS) after protein precipitation.

### Pharmacokinetic Analysis

Model-independent analyses were performed to evaluate the pharmacokinetics. The area under the concentration-time curve (AUC) and the area under the first moment curve (AUMC) were calculated from zero to 6 or 8 hours post-dose using the linear trapezoidal method with extrapolation to infinity. The terminal elimination rate constant (kₑₗ) and half-life (t_{1/2}) were calculated by linear least-squares regression of log-transformed concentration-time data. Systemic plasma clearance (CLs) was computed from Dose/AUC. Volume of distribution at steady state (Vdss) was calculated from Dose·AUMC/AUC². WinNonlin® Professional software (Pharsight Corporation) was used to generate all pharmacokinetic data.

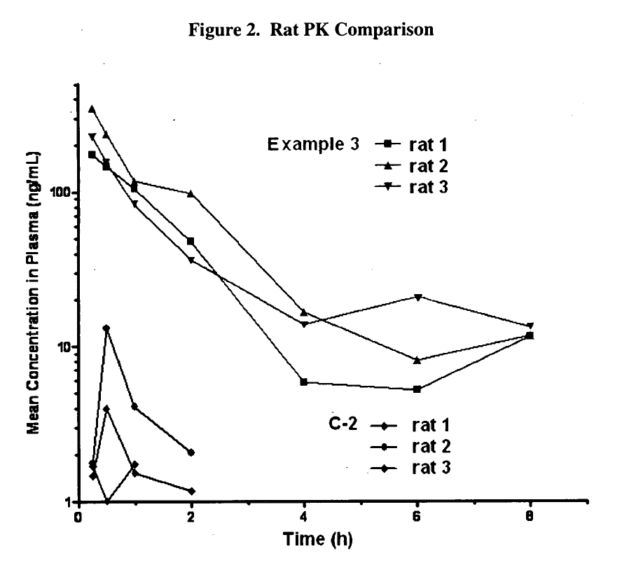

Results of the pharmacokinetic studies in rats are depicted graphically in FIG. 2. As shown in FIG. 2 in the three rats tested, Example 3 demonstrated consistently higher plasma drug exposures, less variability in plasma exposure and time course, and an approximately 8-fold improvement in oral bioavailability (F=8%) as compared to Example C-2 (F˜1%). The improved pharmacokinetic properties of Example 3 as compared to the pharmacokinetic properties of C-2 were surprising and unexpected.

Example 2 was also studied in rats and demonstrated similarly improved in vivo pharmacokinetic behavior (F=11%).

### B. Dog PK Protocol

### Dose Formulation

The PO dose formulations were prepared on the day of dosing by dissolving 33.16 mg of test material in 60 mL of sterile water to achieve a concentration of 0.5526 mg/mL total compound (0.5 mg/mL test compound). The dosing solution was vortexed to ensure homogeneity and dissolution.

### Dose Administration

The dose formulation was administered via oral gavage per facility SOPs. Each dog received a single PO dose of 6 mg/kg.

### Blood Collection

All blood samples were collected from a peripheral vessel. Approximately 0.3 mL blood was collected at each time point. All blood samples were placed on wet ice until processed for plasma.

### Blood/Plasma Processing

Blood samples were processed for plasma by centrifugation at approximately 5° C. Plasma samples were stored in polypropylene tubes, quick frozen over dry ice and kept at −70±10° C. until LC/MSMS analysis.

### Sample Analysis

Plasma concentrations were determined by high performance liquid chromatography with tandem mass spectrometric detection (LC/MS/MS) after protein precipitation.

### Data Analysis

Plasma concentration versus time data was analyzed by non-compartmental approaches using the WinNonlin software program (version 5.0.1, Pharsight, Mountain View, Calif.).

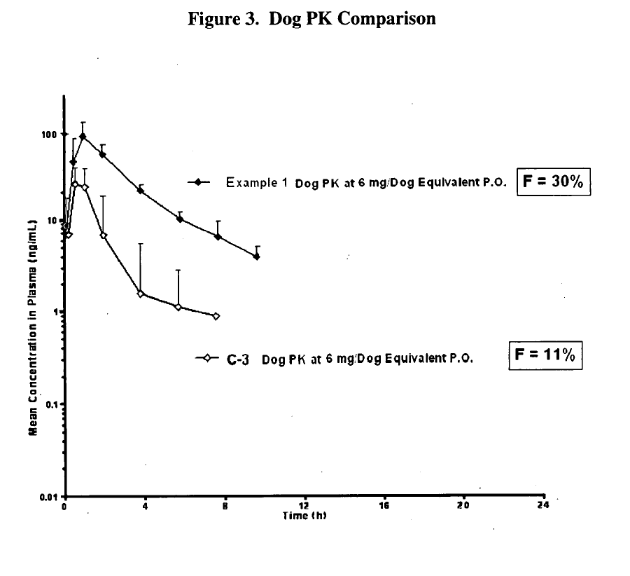

Results of pharmacokinetic studies in dogs are depicted graphically in FIG. 3. As shown in FIG. 3 for the three dogs tested, Example 1 demonstrated consistently higher plasma drug exposures, clearly improved oral bioavailability (F=30%), and less variability in plasma exposure and time course, particularly at time points between 1-6 h (as evidenced by smaller error bars), compared to Example C-3 (F=11%). Example 1 also demonstrated higher bioavailability (specifically, an approximately 3-fold increase in mean concentration in plasma) as compared to C-3. The calculated AUC values for each dog are set forth in Table III below.

TABLE III

| | | Dose | |
|---|---|---|---|
| Example | Animal # | (mg/kg) | AUC0-t (ng*h/mL) |
| 1 | Dog #1 | 6 | 183 |
| 1 | Dog #2 | 6 | 250 |
| 1 | Dog #3 | 6 | 344 |
| C-3 | Dog #1 | 6 | 54 |
| C-3 | Dog #2 | 6 | 53 |
| C-3 | Dog #3 | 6 | 81 |

Analysis of the data in Table III shows that there was an approximately 4-fold improvement in the mean AUC value for Example 1 (AUC=259±81 ng*h/ml) as compared to Example C-3 (AUC=63±16 ng*h/ml). The improved pharmacokinetic properties of Example 1 as compared to the pharmacokinetic properties of prior art compound C-3 were surprising and unexpected.

### B. Primate PK Protocol

### Dose Formulation

The dose formulations were prepared on the day of dosing by dissolving 33.16 mg of test material in 60 mL of sterile water to achieve a concentration of 0.5526 mg/mL total compound (0.5 mg/mL test compound). The dosing solution was vortexed to ensure homogeneity and dissolution.

### Dose Administration

The dose formulation was administered via oral gavage per facility SOPs. Each monkey received a single PO dose of 1 mg/kg as a 0.5% MC suspension.

### Blood Collection:

All blood samples were collected from a peripheral vessel. Approximately 0.3 mL blood was collected at each time point. All blood samples were placed on wet ice until processed for plasma.

### Blood/Plasma Processing

Blood samples were processed for plasma by centrifugation at approximately 5° C. Plasma samples were be stored in polypropylene tubes, quick frozen over dry ice and kept at −70±10° C. until LC/MSMS analysis.

### Sample Analysis

Plasma concentrations were determined by high performance liquid chromatography with tandem mass spectrometric detection (LC/MS/MS) after protein precipitation.

### Data Analysis

Plasma concentration versus time data was analyzed by non-compartmental approaches using the WinNonlin software program (version 5.0.1, Pharsight, Mountain View, Calif.).

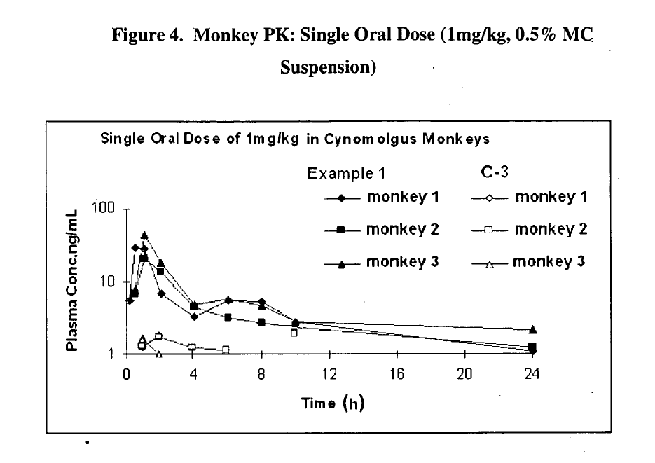

Results of pharmacokinetic studies in monkeys are depicted graphically in FIG. 4. As with the pharmacokinetic studies in rats and dogs, Example 1 demonstrated consistently higher plasma drug exposures, less variability in plasma exposure and time course, and clearly improved oral bioavailability (F=10%) in the three monkeys tested, as evidenced by narrower error bars, compared to Example C-3 (F=<2%). (It should be noted that due to poor bioavailability resulting in limited exposure at the administered dose, precise AUC values for C-3 could not be determined.) Example 1 demonstrated an 18.3-fold higher maximum plasma concentration (Cₘₐₓ) as compared to Example C-3. Specifically, Example 1 demonstrated a Cₘₐₓ of 32±12 ng/mg and Example C-3 demonstrated a Cₘₐₓ of 1.75±0.06 ng/ml. The calculated AUC values for each monkey are set forth in Table IV below.

TABLE IV

| | | Dose | Cmax | Mean Cmax ± | AUC0-t |
|---|---|---|---|---|---|
| Example | Animal # | (mg/kg) | (ng/mL) | SD (ng/mL) | (ng*h/mL) |
| 1 | Monkey #1 | 1 | 29.5 | 32 ± 12 | 103 |
| 1 | Monkey #2 | 1 | 20.8 | 32 ± 12 | 87 |
| 1 | Monkey #3 | 1 | 44.6 | 32 ± 12 | 132 |
| C-3 | Monkey #1 | 1 | 1.81 | 1.75 ± 0.06 | — |
| C-3 | Monkey #2 | 1 | 1.74 | 1.75 ± 0.06 | — |
| C-3 | Monkey #3 | 1 | 1.70 | 1.75 ± 0.06 | — |

Analysis of the data in Table IV indicates that the Cₘₐₓ values for Example 1 were about 10 to 20-fold greater than Cₘₐₓ values for Example C-3. The improved pharmacokinetic properties of Example 1 as compared to the pharmacokinetic properties of prior art compound C-3 were surprising and unexpected. The results of these studies are particularly advantageous in view of the general predictive nature of primate pharmacokinetics on human pharmacokinetics.

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### Embodiment 1

A compound of Formula I:

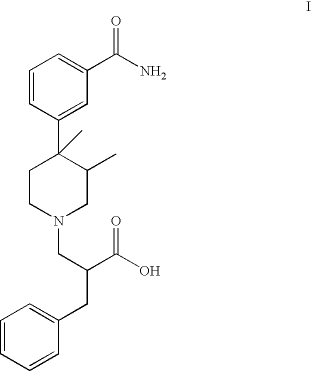

or a salt thereof.

### Embodiment 2

A compound according to Embodiment 1, which is in a non-salt form.

### Embodiment 3

A compound according to Embodiment 1 which is in the form of a salt.

### Embodiment 4

A salt according to Embodiment 3 which is a non-pharmaceutically acceptable salt.

### Embodiment 5

A salt according to Embodiment 3 wherein the salt is a pharmaceutically acceptable salt.

### Embodiment 6

A salt according to Embodiment 3, which has Formula I-S-1, I-S-2 or I-S-3:

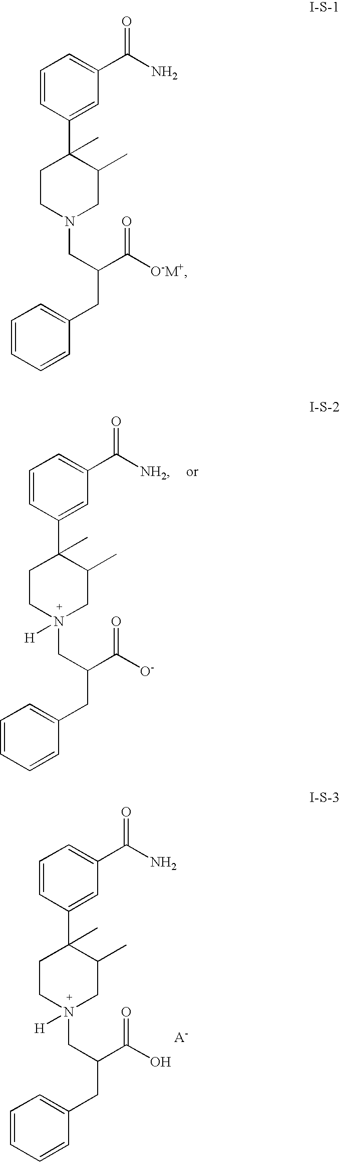

wherein:
M⁺ is a cation of a base; and
A⁻ is an anion of an acid.

### Embodiment 7

A salt according to Embodiment 6 which has Formula I-S-1.

### Embodiment 8

A salt according to Embodiment 7, wherein M⁺ is selected from the group consisting of an ammonium cation and a metal cation.

### Embodiment 9

A salt according to Embodiment 8, wherein M⁺ is a metal cation.

### Embodiment 10

A salt according to Embodiment 9, wherein said metal cation is selected from the group consisting of a sodium cation and a lithium cation.

### Embodiment 11

A salt according to Embodiment 10, wherein said metal cation is a sodium cation.

### Embodiment 12

A salt according to Embodiment 6, which has Formula I-S-2.

### Embodiment 13

A salt according to Embodiment 6, which has Formula IA-S-3.

### Embodiment 14

A salt according to Embodiment 13, wherein A⁻ is a trifluoroacetate ion, succinate ion, or oxalate ion.

### Embodiment 15

A salt according to Embodiment 14, wherein A⁻ is a trifluoroacetate ion.

### Embodiment 16

A salt according to Embodiment 14, wherein A⁻ is a succinate ion.

### Embodiment 17

A salt according to Embodiment 14, wherein A⁻ is an oxalate ion.

### Embodiment 18

A compound or salt thereof according to Embodiment 1, which has a stereochemical configuration selected from the group consisting of RRR, RRS, RSR, SRR, RSS, SRS, SSR, and SSS.

### Embodiment 19

A compound or salt thereof according to Embodiment 18, which has the stereochemical configuration RRR.

### Embodiment 20

A compound or salt thereof according to Embodiment 18, which has the stereochemical configuration RRS.

### Embodiment 21

A compound or salt thereof according to Embodiment 18, which has the stereochemical configuration RSR.

### Embodiment 22

A compound or salt thereof according to Embodiment 18, which has the stereochemical configuration SRR.

### Embodiment 23

A compound or salt thereof according to Embodiment 18, which has the stereochemical configuration RSS.

### Embodiment 24

A compound or salt thereof according to Embodiment 28, which has the stereochemical configuration SRS.

### Embodiment 25

A compound or salt thereof according to Embodiment 18, which has the stereochemical configuration SSR.

### Embodiment 26

A compound or salt thereof according to Embodiment 18, which has a stereochemical configuration SSS.

### Embodiment 27

A compound or salt thereof according to Embodiment 1, wherein the compound of Formula I has the following Formula IA:

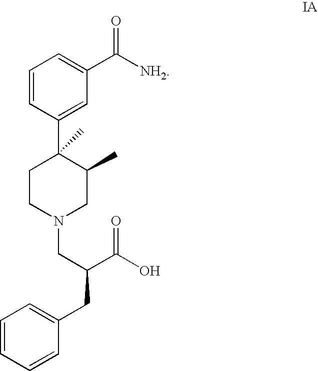

### Embodiment 28

A compound according to Embodiment 27, which is in a non-salt form.

### Embodiment 29

A compound according to Embodiment 27, which is in the form of a salt.

### Embodiment 30

A salt according to Embodiment 29, which is a non-pharmaceutically acceptable salt.

### Embodiment 31

A salt according to Embodiment 29, wherein the salt is a pharmaceutically acceptable salt.

### Embodiment 32

A salt according to Embodiment 29, which has Formula IA-S-1, IA-S-2 or IA-S-3:

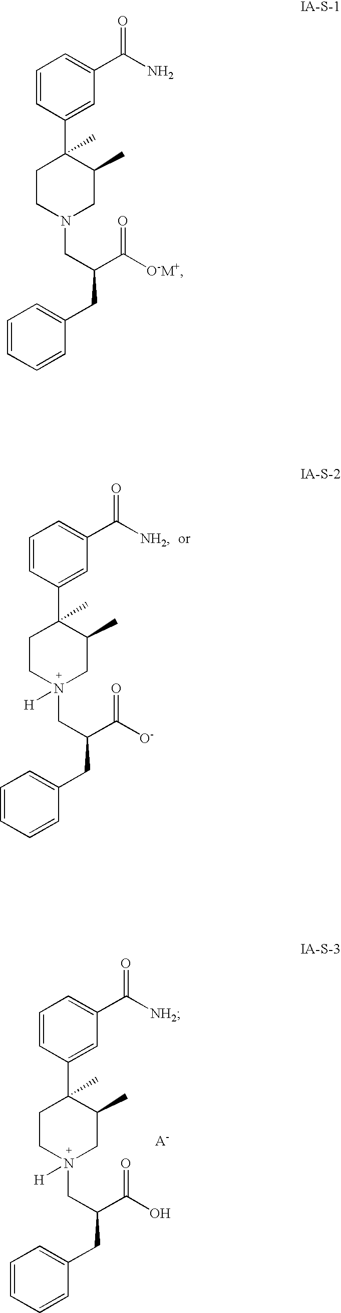

wherein:
M⁺ is a cation of a base; and
A⁻ is an anion of an acid.

### Embodiment 33

A salt according to Embodiment 32, which has Formula IA-S-1.

### Embodiment 34

A salt according to Embodiment 33, wherein M⁺ is selected from the group consisting of an ammonium cation and a metal cation.

### Embodiment 35

A salt according to Embodiment 34, wherein M⁺ is a metal cation.

### Embodiment 36

A salt according to Embodiment 35, wherein said metal cation is selected from the group consisting of a sodium cation and a lithium cation.

### Embodiment 37

A salt according to Embodiment 36, wherein said metal cation is a sodium cation.

### Embodiment 38

A salt according to Embodiment 32, which has Formula IA-S-2.

### Embodiment 39

A salt according to Embodiment 32, which has Formula IA-S-3.

### Embodiment 40

A salt according to Embodiment 39, wherein A⁻ is a trifluoroacetate ion, succinate ion, or oxalate ion.

### Embodiment 41

A salt according to Embodiment 40, wherein A⁻ is a trifluoroacetate ion.

### Embodiment 42

A salt according to Embodiment 40, wherein A⁻ is a succinate ion.

### Embodiment 43

A salt according to Embodiment 40, wherein A⁻ is an oxalate ion.

### Embodiment 44

A pharmaceutical composition comprising:

a pharmaceutically acceptable carrier; and

an effective amount of a compound or pharmaceutically acceptable salt thereof according to any one of Embodiments 1 to 3, 5 to 13, 16 to 29, 31 to 39, 42, or 43.

### Embodiment 45

A pharmaceutical composition according to Embodiment 44, further comprising an effective amount of at least one opioid.

### Embodiment 46

A pharmaceutical composition according to Embodiment 45, wherein the opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol and mixtures thereof.

### Embodiment 47

A method of binding opioid receptors comprising:

contacting the opioid receptors with an effective amount of a compound or salt thereof or pharmaceutical composition according to any one of Embodiments 1 to 46.

### Embodiment 48

A method according to Embodiment 47, comprising binding the opioid receptors in vitro.

### Embodiment 49

A method according to Embodiment 47, comprising binding the opioid receptors in vivo.

### Embodiment 50

A method according to Embodiment 49, which comprises binding opioid receptors in a patient in need thereof comprising:

administering to said patient an effective amount of said compound or salt thereof or pharmaceutical composition according to any one of Embodiments 1 to 3, 5 to 13, 16 to 29, 31 to 39, or 42 to 46.

### Embodiment 51

A method according to any of Embodiments 47 to 50, wherein the receptors are μ opioid receptors.

### Embodiment 52

A method according to Embodiment 49, wherein said μ opioid receptors are located in the central nervous system.

### Embodiment 53

A method according to Embodiment 49, wherein said μ opioid receptors are located peripherally to the central nervous system.

### Embodiment 54

A method according to Embodiment 47, wherein the binding antagonizes the activity of the opioid receptors.

### Embodiment 55

A method according to Embodiment 49, wherein said compound or salt thereof exhibits activity toward the opioid receptors.

### Embodiment 56

A method according to Embodiment 49, wherein said compound or salt thereof does not substantially cross the blood-brain barrier.

### Embodiment 57

A method according to Embodiment 50, wherein the patient is in need of treatment of a condition or disease caused by an opioid.

### Embodiment 58

A method according to Embodiment 57, wherein said opioid is endogenous.

### Embodiment 59

A method according to Embodiment 57, wherein said opioid is exogenous.

### Embodiment 60

A method for treating gastrointestinal dysfunction comprising:

administering to a patient in need of such treatment an effective amount of a compound or salt thereof or pharmaceutical composition according to any one of Embodiments 1 to 3, 5 to 13, 16 to 29, 31 to 39, or 42 to 46.

### Embodiment 61

A method for treating ileus, comprising:

administering to a patient in need of such treatment an effective amount of a compound or salt thereof or pharmaceutical composition according to any one of Embodiments 1 to 3, 5 to 13, 16 to 29, 31 to 39, or 42 to 46.

### Embodiment 62

A method for treating a side effect associated with an opioid comprising:

administering to a patient in need of such treatment an effective amount of a compound or salt thereof or pharmaceutical composition according to any one of Embodiments 1 to 3, 5 to 13, 16 to 29, 31 to 39, or 42 to 46.

### Embodiment 63

A method according to Embodiment 62, which further comprises administering to said patient an effective amount of at least one opioid.

### Embodiment 64

A method according to Embodiment 62, wherein the side effect is selected from the group consisting of constipation, nausea and vomiting.

### Embodiment 65

A method according to Embodiment 62, wherein said administering occurs before, during or after administering at least one opioid.

### Embodiment 66

A method according to Embodiment 63 or 65, wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol and mixtures thereof.

### Embodiment 67

A method of treating pain comprising:

administering to a patient in need thereof, a composition comprising:

an effective amount of an opioid; and

an effective amount of a compound or salt thereof or pharmaceutical composition according to any one of Embodiments 1 to 3, 5 to 13, 16 to 29, 31 to 39, or 42 to 46.

What is claimed:

1. A compound of Formula IA: 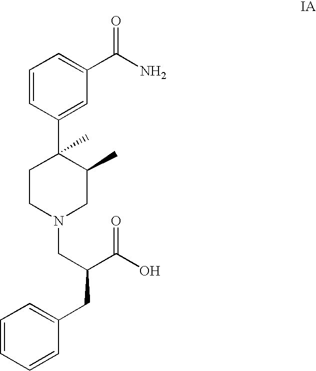
or a salt thereof.

2. A compound according to claim 1, which is in a non-salt form.

3. A compound according to claim 1, which is in the form of a salt.

4. A salt according to claim 3, which is a non-pharmaceutically acceptable salt.

5. A salt according to claim 3 which is a pharmaceutically acceptable salt.

6. A salt according to claim 3, which has Formula IA-S-1, IA-S-2 or IA-S-3: 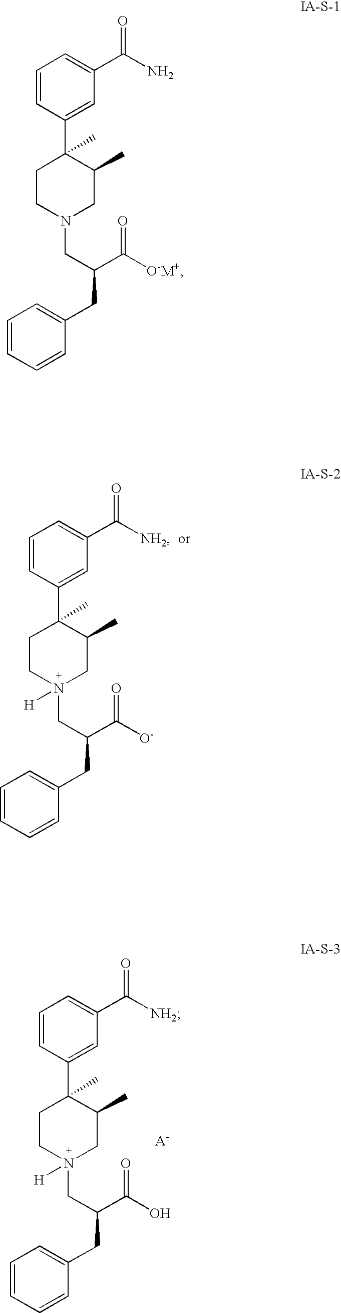
wherein:
M⁺ is a cation of a base; and
A⁻ is an anion of an acid.

7. A salt according to claim 6 which has Formula IA-S-1.

8. A salt according to claim 7, wherein M⁺ is selected from the group consisting of an ammonium cation and a metal cation.

9. A salt according to claim 8, wherein M⁺ is a metal cation.

10. A salt according to claim 9, wherein said metal cation is selected from the group consisting of a sodium cation and a lithium cation.

11. A salt according to claim 10, wherein said metal cation is a sodium cation.

12. A salt according to claim 6, which has Formula IA-S-2.

13. A salt according to claim 6, which has Formula IA-S-3.

14. A salt according to claim 13, wherein A⁻ is a trifluoroacetate ion, succinate ion, or oxalate ion.

15. A salt according to claim 14, wherein A⁻ is a trifluoroacetate ion.

16. A salt according to claim 14, wherein A⁻ is a succinate ion.

17. A salt according to claim 14, wherein A⁻ is an oxalate ion.

18. A pharmaceutical composition, comprising:
a pharmaceutically acceptable carrier; and
an effective amount of a compound of the following Formula IA: 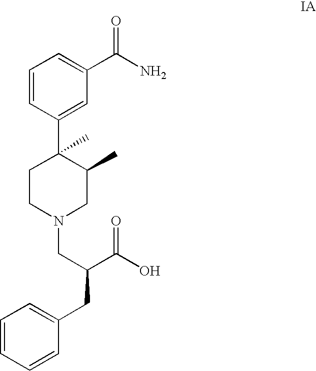
or a pharmaceutically acceptable salt thereof.

19. A pharmaceutical composition according to claim 18, wherein said compound is in the form of a pharmaceutically acceptable salt.

20. A pharmaceutical composition according to claim 19, wherein said pharmaceutically acceptable salt has Formula IA-S-1, IA-S-2 or IA-S-3: 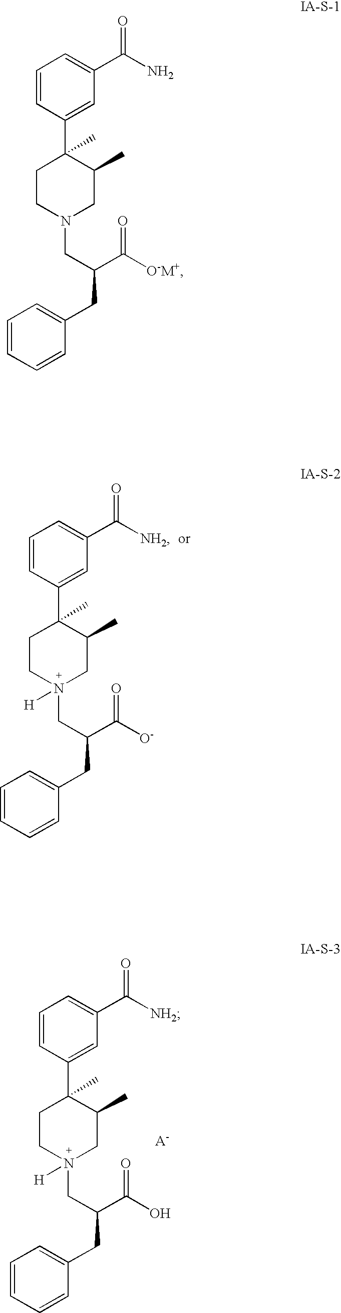
wherein:
M⁺ is a cation of a pharmaceutically acceptable base; and
A⁻ is an anion of a pharmaceutically acceptable acid.

21. A pharmaceutical composition according to claim 20, wherein said pharmaceutically acceptable salt has Formula IA-S-1.

22. A pharmaceutical composition according to claim 21, wherein M⁺ is selected from the group consisting of an ammonium cation and a metal cation.

23. A pharmaceutical composition according to claim 21, wherein M⁺ is a metal cation.

24. A pharmaceutical composition according to claim 23, wherein said metal cation is selected from the group consisting of a sodium cation and a lithium cation.

25. A pharmaceutical composition according to claim 24, wherein said metal cation is a sodium cation.

26. A pharmaceutical composition according to claim 20, wherein said pharmaceutically acceptable salt has Formula IA-S-2.

27. A pharmaceutical composition according to claim 20, wherein said pharmaceutically acceptable salt has Formula IA-S-3.

28. A pharmaceutical composition according to claim 27, wherein A⁻ is a succinate ion or oxalate ion.

29. A pharmaceutical composition according to claim 28, wherein A⁻ is a succinate ion.

30. A pharmaceutical composition according to claim 28, wherein A⁻ is an oxalate ion.

31. A pharmaceutical composition according to claim 18, further comprising an effective amount of at least one opioid.

32. A pharmaceutical composition according to claim 31, wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol and mixtures thereof.

33. A method of binding opioid receptors comprising:
contacting the opioid receptors with an effective amount of a compound or salt thereof according to claim 1.

34. A method according to claim 33, wherein said compound is in a non-salt form.

35. A method according to claim 33, wherein said compound is in the form of a salt.

36. A method according to claim 35, wherein said salt is a non-pharmaceutically acceptable salt.

37. A method according to claim 35 which is a pharmaceutically acceptable salt.

38. A method according to claim 33, wherein the receptors are μ opioid receptors.

39. A method according to claim 33, wherein the binding antagonizes the activity of the opioid receptors.

40. A method according to claim 33, comprising binding the opioid receptors in vitro.

41. A method according to claim 35, wherein said salt has Formula IA-S-1, IA-S-2, or IA-S-3: 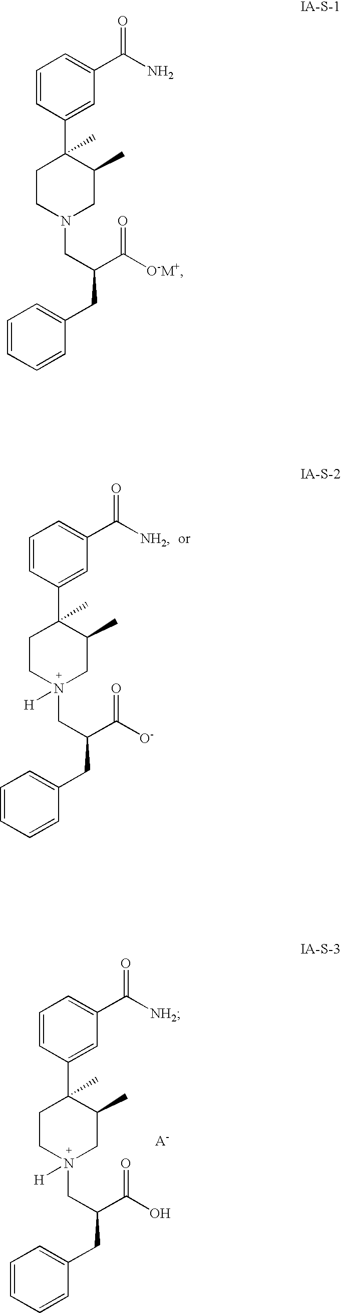
wherein:
M⁺ is a cation of a base; and
A⁻ is an anion of an acid.

42. A method according to claim 41, wherein said salt has Formula IA-S-1.

43. A method according to claim 42, wherein M⁺ is selected from the group consisting of an ammonium cation and a metal cation.

44. A method according to claim 43, wherein M⁺ is a metal cation.

45. A method according to claim 44, wherein said metal cation is selected from the group consisting of a sodium cation and a lithium cation.

46. A method according to claim 45, wherein said metal cation is a sodium cation.

47. A method according to claim 35, wherein said salt has Formula IA-S-2.

48. A method according to claim 35, wherein said salt has Formula IA-S-3.

49. A method according to claim 48, wherein A⁻ is a trifluoroacetate ion, succinate ion, or oxalate ion.

50. A method according to claim 49, wherein A⁻ is a trifluoroacetate ion.

51. A method according to claim 49, wherein A⁻ is a succinate ion.

52. A method according to claim 49, wherein A⁻ is an oxalate ion.

53. A method according to claim 33, comprising binding the opioid receptors in vivo.

54. A method according to claim 53, which comprises binding opioid receptors in a patient in need thereof comprising:
administering to said patient an effective amount of said compound or a pharmaceutically acceptable salt thereof.

55. A method according to claim 54, wherein the receptors are μ opioid receptors.

56. A method according to claim 55, wherein said μ opioid receptors are located in the central nervous system.

57. A method according to claim 55, wherein said μ opioid receptors are located peripherally to the central nervous system.

58. A method according to claim 54, wherein the binding antagonizes the activity of the opioid receptors.

59. A method according to claim 54, wherein said compound or pharmaceutically acceptable salt thereof exhibits activity toward the opioid receptors.

60. A method according to claim 54, wherein said compound or pharmaceutically acceptable salt thereof does not substantially cross the blood-brain barrier.

61. A method according to claim 54, wherein the patient is in need of treatment of a condition or disease caused by an opioid.

62. A method according to claim 61, wherein said opioid is endogenous.

63. A method according to claim 61, wherein said opioid is exogenous.

64. A method according to claim 54, wherein said compound is administered to the patient in a non-salt form.

65. A method according to claim 54, wherein said compound is administered to the patient in the form of a pharmaceutically acceptable salt.

66. A method according to claim 65, wherein said pharmaceutically acceptable salt has Formula IA-S-1, IA-S-2, or IA-S-3: 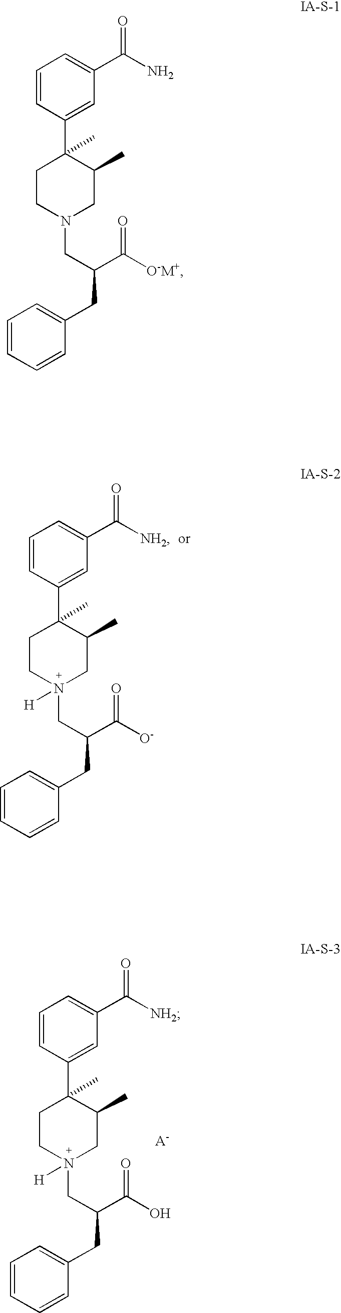
wherein:
M⁺ is a cation of a pharmaceutically acceptable base; and
A⁻ is an anion of a pharmaceutically acceptable acid.

67. A method according to claim 66, wherein said pharmaceutically acceptable salt has Formula IA-S-1.

68. A method according to claim 67, wherein M⁺ is selected from the group consisting of an ammonium cation and a metal cation.

69. A method according to claim 68, wherein M⁺ is a metal cation.

70. A method according to claim 69, wherein said metal cation is selected from the group consisting of a sodium cation and a lithium cation.

71. A method according to claim 70, wherein said metal cation is a sodium cation.

72. A method according to claim 66, wherein said pharmaceutically acceptable salt has Formula IA-S-2.

73. A method according to claim 66, wherein said pharmaceutically acceptable salt has Formula IA-S-3.

74. A method according to claim 73, wherein A⁻ is a succinate ion or oxalate ion.

75. A method according to claim 74, wherein A⁻ is a succinate ion.

76. A method according to claim 74, wherein A⁻ is an oxalate ion.

77. A method for treating gastrointestinal dysfunction comprising:
administering to a patient in need of such treatment an effective amount of a compound of Formula IA: 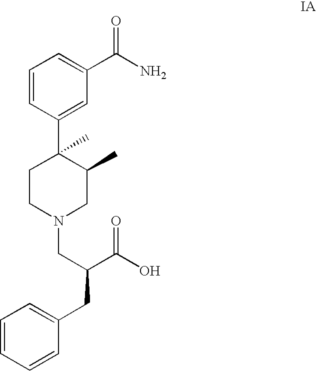
or a pharmaceutically acceptable salt thereof.

78. A method according to claim 77, wherein said compound is administered to said patient in a non-salt form.

79. A method according to claim 77, wherein said compound is administered to the patient in the form of a pharmaceutically acceptable salt.

80. A method according to claim 79, wherein said pharmaceutically acceptable salt has Formula IA-S-1, IA-S-2, or IA-S-3: 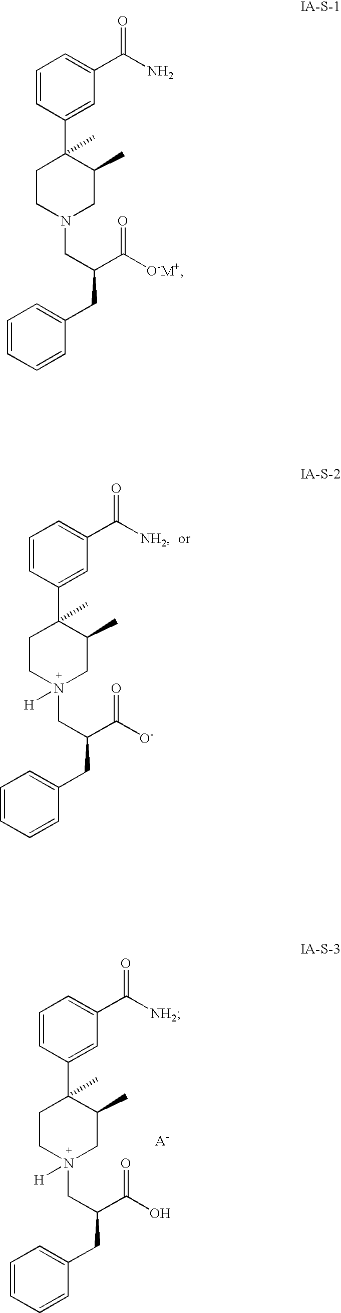
wherein:
M⁺ is a cation of a pharmaceutically acceptable base; and
A⁻ is an anion of a pharmaceutically acceptable acid.

81. A method according to claim 80, wherein said pharmaceutically acceptable salt has Formula IA-S-1.

82. A method according to claim 81, wherein M⁺ is selected from the group consisting of an ammonium cation and a metal cation.

83. A method according to claim 82, wherein M⁺ is a metal cation.

84. A method according to claim 83, wherein said metal cation is selected from the group consisting of a sodium cation and a lithium cation.

85. A method according to claim 80, wherein said pharmaceutically acceptable salt has Formula IA-S-2.

86. A method according to claim 80, wherein said pharmaceutically acceptable salt has Formula IA-S-3.

87. A method according to claim 86, wherein A⁻ is a succinate ion or oxalate ion.

88. A method according to claim 87, wherein A⁻ is a succinate ion.

89. A method according to claim 87, wherein A⁻ is an oxalate ion.

90. A method for treating ileus comprising:
administering to a patient in need of such treatment an effective amount of a compound of Formula IA: 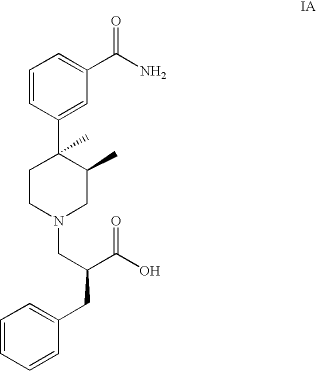
or a pharmaceutically acceptable salt thereof.

91. A method according to claim 90, wherein said compound is administered to said patient in a non-salt form.

92. A method according to of claim 90, wherein said compound is administered to the patient in the form of a pharmaceutically acceptable salt.

93. A method according to claim 92, wherein said pharmaceutically acceptable salt has Formula IA-S-1, IA-S-2, or IA-S-3: 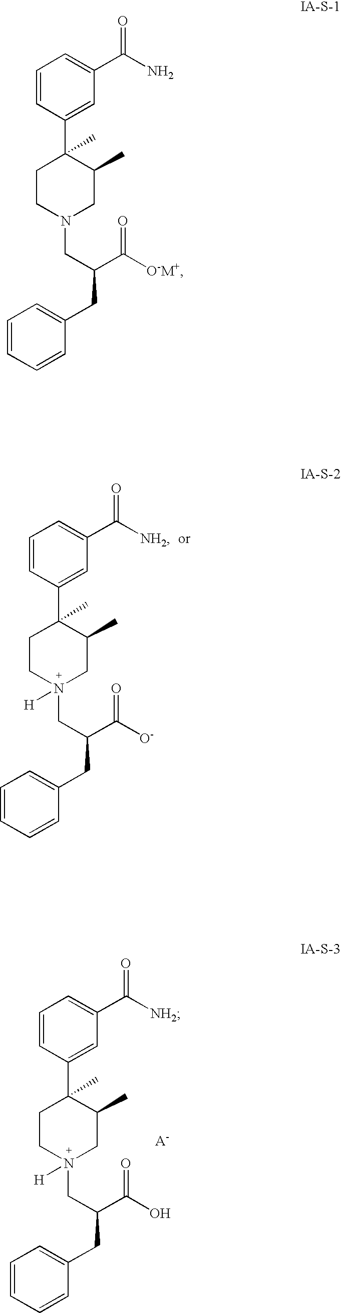
wherein:
M⁺ is a cation of a pharmaceutically acceptable base; and
A⁻ is an anion of a pharmaceutically acceptable acid.

94. A method according to claim 93, wherein said pharmaceutically acceptable salt has Formula IA-S-1.

95. A method according to claim 94, wherein M⁺ is selected from the group consisting of an ammonium cation and a metal cation.

96. A method according to claim 95, wherein M⁺ is a metal cation.

97. A method according to claim 95, wherein said metal cation is selected from the group consisting of a sodium cation and a lithium cation.

98. A method according to claim 97, wherein said metal cation is a sodium cation.

99. A method according to claim 93, wherein said pharmaceutically acceptable salt has Formula IA-S-2.

100. A method according to claim 93, wherein said pharmaceutically acceptable salt has Formula IA-S-3.

101. A method according to claim 100, wherein A⁻ is a succinate ion or oxalate ion.

102. A method according to claim 101, wherein A⁻ is a succinate ion.

103. A method according to claim 101, wherein A⁻ is an oxalate ion.

104. A method for treating a side effect associated with an opioid comprising:
administering to a patient in need of such treatment an effective amount of a compound of Formula IA: 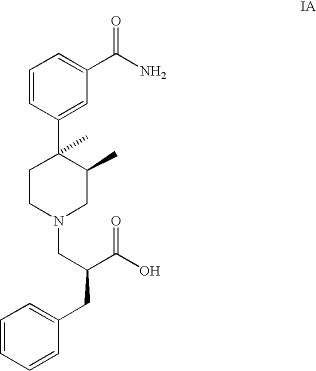
or a pharmaceutically acceptable salt thereof.

105. A method according to claim 104, wherein said compound is administered to said patient in a non-salt form.

106. A method according to of claim 104, wherein said compound is administered to the patient in the form of a pharmaceutically acceptable salt.

107. A method according to claim 104, wherein said pharmaceutically acceptable salt has Formula IA-S-1, IA-S-2, or IA-S-3: 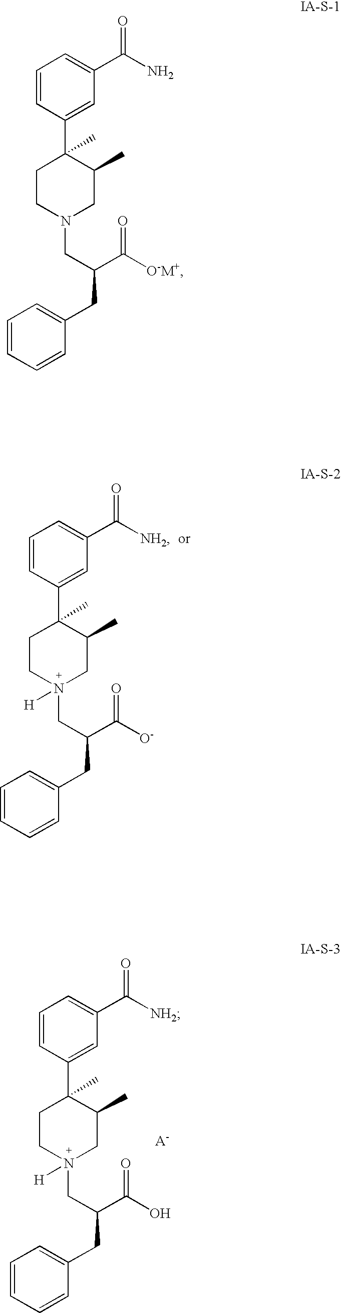
wherein:
M⁺ is a cation of a pharmaceutically acceptable base; and
A⁻ is an anion of a pharmaceutically acceptable acid.

108. A method according to claim 107, wherein said pharmaceutically acceptable salt has Formula IA-S-1.

109. A method according to claim 108, wherein M⁺ is selected from the group consisting of an ammonium cation and a metal cation.

110. A method according to claim 109, wherein M⁺ is a metal cation.

111. A method according to claim 110, wherein said metal cation is selected from the group consisting of a sodium cation and a lithium cation.

112. A method according to claim 111, wherein said metal cation is a sodium cation.

113. A method according to claim 107, wherein said pharmaceutically acceptable salt has Formula IA-S-2.

114. A method according to claim 107, wherein said pharmaceutically acceptable salt has Formula IA-S-3.

115. A method according to claim 114, wherein A⁻ is a succinate ion or oxalate ion.

116. A method according to claim 115, wherein A⁻ is a succinate ion.

117. A method according to claim 115, wherein A⁻ is an oxalate ion.

118. A method according to claim 104, which further comprises administering to said patient an effective amount of at least one opioid.

119. A method according to claim 104, wherein the side effect is selected from the group consisting of constipation, nausea and vomiting.

120. A method according to claim 104, wherein said administering occurs before, during or after administering at least one opioid.

121. A method according to claim 118, wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol and mixtures thereof.

122. A method of treating pain comprising:
administering to a patient in need thereof, a composition comprising:
an effective amount of an opioid; and
an effective amount of a compound of Formula IA: 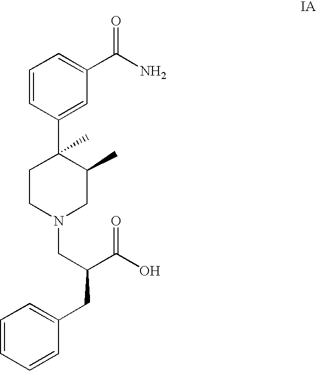
or a pharmaceutically acceptable salt thereof.

123. A method according to claim 122, wherein said compound is administered to said patient in a non-salt form.

124. A method according to claim 122, wherein said compound is administered to the patient in the form of a pharmaceutically acceptable salt.

125. A method according to claim 124, wherein said pharmaceutically acceptable salt has Formula IA-S-1, IA-S-2, or IA-S-3: 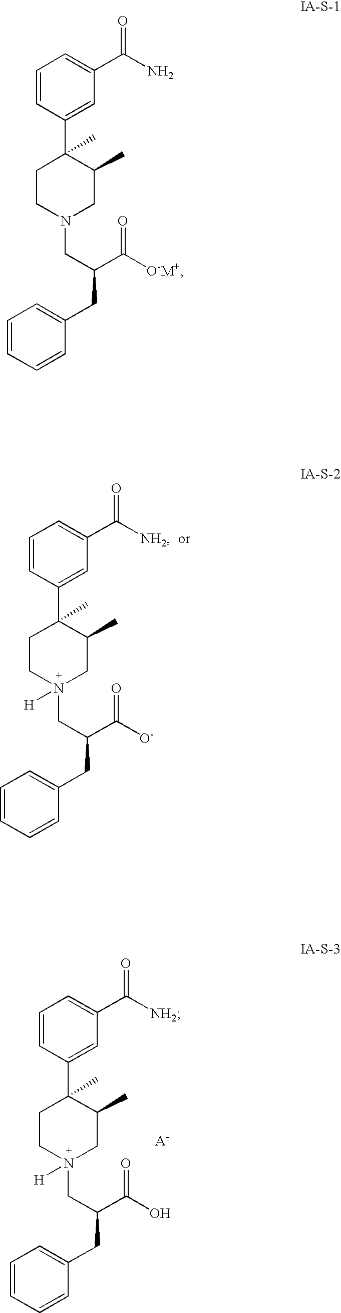
wherein:
M⁺ is a cation of a pharmaceutically acceptable base; and
A⁻ is an anion of a pharmaceutically acceptable acid.

126. A method according to claim 125, wherein said pharmaceutically acceptable salt has Formula IA-S-1.

127. A method according to claim 126, wherein M⁺ is selected from the group consisting of an ammonium cation and a metal cation.

128. A method according to claim 126, wherein M⁺ is a metal cation.

129. A method according to claim 126, wherein said metal cation is selected from the group consisting of a sodium cation and a lithium cation.

130. A method according to claim 129, wherein said metal cation is a sodium cation.

131. A method according to claim 125, wherein said pharmaceutically acceptable salt has Formula IA-S-2.

132. A method according to claim 125, wherein said pharmaceutically acceptable salt has Formula IA-S-3.

133. A method according to claim 132, wherein A⁻ is a succinate ion or oxalate ion.

134. A method according to claim 133, wherein A⁻ is a succinate ion.

135. A method according to claim 133, wherein A⁻ is an oxalate ion.

136. A method according to claim 120, wherein said opioid is selected from the group consisting of alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, tramadol and mixtures thereof.
